(19) United States
(12) Patent Application Publication
(10) Pub. No.: US 2009/0287283 A1
(43) Pub. Date: Nov. 19, 2009

(54) Thermal compress assembly and system with external frame

(76) Inventors: Seth Biser, Fleetwood, NY (US); Lawrence Thad Levine, Easton, CT (US); Harvey Levine, Fairfield, CT (US); William Finneran, New York, NY (US)

Correspondence Address: COOLEY GODWARD KRONISH LLP;ATTN: Patent Group, Suite 1100, 777 - 6th Street, NW, WASHINGTON, DC, 20001, US

(21) Appl. No.: 12/153,322

(22) Filed: May 16, 2008

(51) Int. Cl. A61F 7/02

(52) U.S. Cl. 607/109

(57) ABSTRACT

A thermally adjustable compress assembly, kit, device and methods of using the same. A compress assembly includes a thermally adjustable gel pack and an external frame positionable against the outwardly facing surface of the gel pack. The external frame can be passively positionable against the gel pack to apply backward pressure against the gel pack during use or can be actively attached to the gel pack to provide additional support to at least a portion of the gravitational weight of the gel pack during use.

### CROSS-REFERENCE TO RELATED APPLICATIONS

The present application incorporates by reference U.S. application No. ______ filed on ______ entitled “THERMAL BODILY COMPRESS KITS AND METHODS OF USING SAME” (Attorney Docket No. 14414/2).

### TECHNICAL FIELD

The present invention is directed to thermal body compress devices, kits, assemblies, systems, and methods of using the same. In a particular application, the present invention is directed to providing therapeutic benefit to a user's eye region.

### BACKGROUND

Both hot and cold compresses play an important role in treating various physical problems. As an illustrative example, not meant to be limiting, one specific area of such problems relates to ocular discomfort and disease. In this example, eye care practitioners have recommended hot compress therapy for various eye conditions including certain types of dry eye syndrome, “styes,” orbital and preseptal cellulitis, acute dacryocystitis, and other conditions. Hot compresses to the eyelids and periorbita are also used for certain postsurgical states, for the promotion of feelings of relaxation, for certain cosmetic or dermatological treatments, and for various other reasons. Cold or cool compresses have been recommended for postoperative states following eye surgery, for symptomatic relief of irritating conditions, for relief of migraines, to promote feelings of relaxation, to allow the application of topical skin therapies for cosmetic and dermatologic treatments, and for various other reasons. In addition to professionally-encouraged use, user-directed self-administration of both hot and cold compresses has been fairly widespread.

In the most common and traditional method of compress therapy, the user holds a washcloth either under hot or cold running tap water, or in a basin of hot or cold water, and then applies the moist, temperature-adjusted washcloth to the body part. This method is popular because washcloths are low in cost and widely available, they are reasonably soft in texture, and their temperature can usually be determined by the user. In addition, the washcloth method allows the user to select how the external pressure is applied against the body part. The specific case of eye compresses is illustrative. Because the eyes are one of the most sensitive and delicate of bodily tissues, most users of the washcloth method will avoid putting pressure directly on the round globe of the eye (the eyeball), and will instead press the washcloth gently into other areas such as the corners of the eyes. The washcloth thereby passively conforms to the round globe of the eye in a safe and comfortable way. Therefore, the washcloth method has been viewed as being particularly useful for hot compress therapy.

However, the washcloth method has numerous disadvantages. The washcloth's temperature decays relatively quickly necessitating frequent re-heatings or re-coolings, especially if the washcloth is wrung out after immersion in water. In the case of compress therapy applied to the eyes or other specific head regions, the washcloth may drape uncomfortably over the face and, if too wet, will tend to drip down the user's arm as the user stands at the sink. Repeated use on a body part of a washcloth left in a bathroom, especially when the bathroom is shared by more than one person, may be unhygienic.

There are also some description of using a gel pack for thermal therapy to a specific body region. Most prior gel packs use straps or bands (typically with elastic properties) to hold these gel packs in proper relation to the body part being treated. The nature of straps or bands is such that, in order to secure the gel pack at its proper location on the body, the strap or band must impart a compressive force from the gel pack against the body part. Should the user find the degree of compressive force uncomfortably high, and aim to decrease it by loosening the strap or band, the user risks loosening the compress so that it slides or tips out of position on the body part, as it is pulled downward by gravity.

Some gel packs achieve proper body positioning through adhesives that attach a gel pack to the user's skin. These systems would typically only allow for one-time use because of the nature of such adhesives. An adhesive-based system would not allow users easily to thermally adjust the gel pack during treatment (for example, by removing it, reheating it, and reapplying it).

In the specific example of eye compress therapy, if too much pressure is placed on the eyes during treatment, the result can range from subjective discomfort to an exacerbation of eye conditions such as glaucoma, uveitis, retinal tears, recurrent erosion syndrome, and other diseases. Excessive eye pressure can also lead to non-arteritic ischemic optic neuropathy, uveitis, and corneal abrasions, and can be especially problematic following certain postoperative states.

A specific challenge faced by compress therapy in the ocular region is to provide a compress that achieves sufficient close contact with the eyes and ocular areas such that the thermal effect of the compress is in sufficient communication with the target tissue but so that the compress does not exert an overly-firm degree of pressure on the eyes and ocular areas. It is desirable that this outcome be achieved in a manner that is convenient for the user; for example, that it be achieved in the sitting-up position. The use of a gel pack for ocular use which is simply attached to the face with a strap or band will not achieve this objective, because a gel pack containing sufficient volume and mass for prolonged compress therapy will tend to be heavy enough to slide downward out of position unless it is strapped firmly in position. However, if such a gel pack is strapped firmly in position, the pressure exerted on the globes of the eyes will tend to be uncomfortable or pose health risks for the user.

None of the current ocular compress systems provides the user with a thermally adjustable gel pack that will maintain a stable position in front of or against the eyes without excess pressure being placed against the eyes by the stabilizing mechanism, can be gently and carefully pressed into position by the user so that it conforms to and maintains the shape of the user's eyes, can be applied with the user in an upright position, can be molded to put extra pressure where desired by the user, and exerts a degree of pressure that exactly matches the user's sensitivity.

### SUMMARY

The present invention provides thermally adjustable body compress devices, assemblies, kits, systems, and methods of preparing and using the same. The body compress assemblies, systems, and kits can be used to provide symptomatic relief of bodily symptoms or to otherwise improve the user's condition.

In an embodiment, the present invention provides a body compress system or assembly comprising a thermal bodily compress and frame assembly. The thermal bodily compress comprises a thermally-adjustable gel pack that is configured to be applied against a body region of a user's body. The gel pack comprises a casing defining a chamber holding a thermally-activatable gelatinous substance. The body compress system or assembly further comprises a frame assembly comprising an external frame. The external frame is attachable to or otherwise positionable against the outwardly facing side of the gel pack (i.e. the side of the gel pack that will not be in contact with the patient's body region in an applied position of the gel pack). Specifically, the external frame is passively positionable on the outwardly facing surface of the gel pack to form a body compress system or actively positionable on the outwardly facing surface of the gel pack to form a body compress assembly. In the former embodiment, the external frame includes a strap to compress the gel pack against the user's anatomy. In the latter embodiment, the gel pack or the external frame includes a strap and the external frame is fastened to the gel pack to support at least a portion of the gravitational weight of the gel pack so that in an applied position the gel pack does not sag to such a degree that the gel pack is no longer able to provide therapeutic benefit to the user. Such an embodiment can generally aid in minimizing the movement of the gel pack relative to the user's body portion in a lateral direction as well as in a superior to inferior direction. In certain embodiments the external frame is fabricated from a material stiff enough to perform this function without buckling when the external frame is in a vertical orientation and the gel pack is secured against the eye region of the user.

Furthermore, in certain embodiments where the external frame is actively attached to the gel pack, at least the top portion of the gel pack is attached to at least the top portion of the external frame and a strap is attached to the right and left sides of the external frame such that tightening or loosening of the strap exerts a controllable horizontal pressure on the external frame which is largely independent from the vertical support provided by the external frame to the gel pack. Such an embodiment allows, among other things, for the user to keep the thermal compress assembly in contact with the body with only a minimally compressive effect.

In certain embodiments, the external frame defines relief openings that provide relief from the direct compressive pressure that would otherwise be transmitted through the external frame at the locations of the openings. Such openings also allow the user to manipulate the gel pack in areas the gel pack directly underlies in order to achieve a precisely-directed therapeutic effect.

In certain embodiments, the present invention provides a body compress kit comprising the above-mentioned components and further comprises a plurality of sheets, wherein each sheet is configured to be applied between the user's body part and the gel pack. The sheets can be fabricated from any biocompatible material. In a preferred embodiment, the sheets are non-woven fabric sheets, foam sheets or film sheets.

In a preferred embodiment, the sheets are non-woven fabric sheets, moistened, disposable, removable from the outer surface of the gel pack, or any combination of the aforementioned features. As with the external frame, the sheets can be passively or actively positionable against the gel pack.

The compress assemblies and systems can be thermally activated by exposure to cold or heat. For example, the compress assemblies and systems could be placed in a refrigerator or freezer, exposed to a cold water or ice bath or exposed to another cold source. The compress assemblies and systems could be exposed to heat by microwave irradiation, a hot water bath or other heat source.

### BRIEF DESCRIPTION OF THE DRAWINGS

FIG. 1 is a schematic illustration in perspective view of an exemplary gel pack according to an embodiment of the present invention.

FIG. 2 is a front view of an exemplary gel pack according to an embodiment of the present invention.

FIG. 3 is a front view of an exemplary gel pack according to an embodiment of the present invention.

FIG. 3A is a front view of an exemplary gel pack according to an embodiment of the present invention.

FIG. 4 is a schematic illustration of an average male human face for purposes of illustrating certain anatomical landmarks and distances therebetween.

FIG. 5 is a front view of an exemplary gel pack according to an embodiment of the present invention.

FIG. 6 is a perspective view of an exemplary gel pack according to an embodiment of the present invention.

FIG. 7 is a front view of an exemplary gel pack according to an embodiment of the present invention.

FIG. 7A is a front view of an exemplary gel pack according to an embodiment of the present invention.

FIG. 8 is a side view illustrating an exemplary gel pack in a resting position according to an embodiment of the present invention.

FIG. 9A is a side view of a gel pack having a bulging configuration in a resting position.

FIG. 9B is a side view of a gel pack having a bulging configuration in an applied position.

FIG. 10 is a front view of an external frame according to an embodiment of the present invention.

FIG. 11 is a front view of an external frame according to an embodiment of the present invention.

FIG. 12A is a plan view of an external frame having a generally planar configuration in a resting position.

FIG. 12B is a plan view of an external frame having a concave surface in a resting position.

FIG. 13A is a schematic illustration of an assembled eye compress assembly in an applied position according to an embodiment of the present invention.

FIG. 13B is a schematic illustration of an assembled eye compress assembly in an applied position according to an embodiment of the present invention.

FIG. 14 is a front view of an external frame according to an embodiment of the present invention.

FIG. 15A is side view of a fastener that includes a button according to an embodiment of the present invention.

FIG. 15B is a side view of an assembled eye compress assembly using the fastener of FIG. 15A.

FIG. 16 is a side view of a fastener according to an embodiment of the present invention.

FIG. 17 is a front view of a gel pack according to an embodiment of the present invention.

FIG. 18 is a perspective view of an external frame and the gel pack of FIG. 17 according to an embodiment of the present invention.

FIG. 19 is a schematic illustration of an assembled eye compress assembly in an applied position according to an embodiment of the present invention.

FIG. 20 is a schematic illustration of an assembled eye compress system in an applied position according to an embodiment of the present invention.

FIG. 21 is a front view of an external frame according to an embodiment of the present invention.

FIG. 22 is a front view of an exemplary sheet according to an embodiment of the present invention.

FIG. 23 is a front view of an external frame and gel impresser according to an embodiment of the present invention.

### DETAILED DESCRIPTION

The present invention is directed to thermally adjustable body compress devices, assemblies, kits, systems, and methods of preparing and using the same. The devices and methods can be used to treat or alleviate a variety of abnormal physiological conditions in users or to provide therapeutic benefit to users who are otherwise in normal condition. The devices and methods can be applied to various body parts such as, for example, the soft tissues, muscles, bones, and other tissues and organs of a user. Non-limiting examples of anatomical sites that devices and methods can be used for include the knee, ankle, and other parts of the leg; the shoulder; the neck; the ears; the back including the lumbar and cervical regions as well as other areas of the back; the face, including the nose and nasal region, the jaw and eye region; and the perineal region. Although the present invention will be described with relation to applying the compress devices and methods to an eye region, it is understood that the invention has broader application to other parts of the anatomy, including those specifically listed above. As used herein, the term “user” includes mammalian subjects including humans.

At a minimum, an eye region of a user that is treated by devices and methods of the present invention includes the periocular region. According to the present invention, the periocular region is defined as including the eyelid, including the skin of the upper and lower eyelids; the eyelid margins; and the lateral canthus and the medial canthus. The periocular region can, but is not required to, include the region of skin directly overlying the ethmoid sinus. In other embodiments, the eye region includes the periorbital region. According to the present invention, the periorbital region includes the eyebrow; either or both of at least a portion of the skin overlying the frontal sinus and at least a portion of the skin overlying the maxillary sinus; at least a portion of the upper cheek; the bridge of the nose and at least a portion of the temple of the head. In certain embodiments, the periorbital region includes the eyebrow, the skin overlying the entire frontal sinus, the skin overlying the entire maxillary sinus, the entire upper check, and the entire temple. In other embodiments, the eye region includes both the periocular region and the periorbital region. Of course, the above described anatomical sites are described in the singular tense but it is understood that these regions are bilateral and thus embodiments of the present invention also cover both the left and right periocular and/or periorbital regions. Of course, in certain embodiments, only the left or right eye region is covered. In other embodiments, the eye region includes the entire temple(s) of the head. In certain embodiments, the eye region includes only one or more of the above-described regions (i.e. does not include the entire face or head).

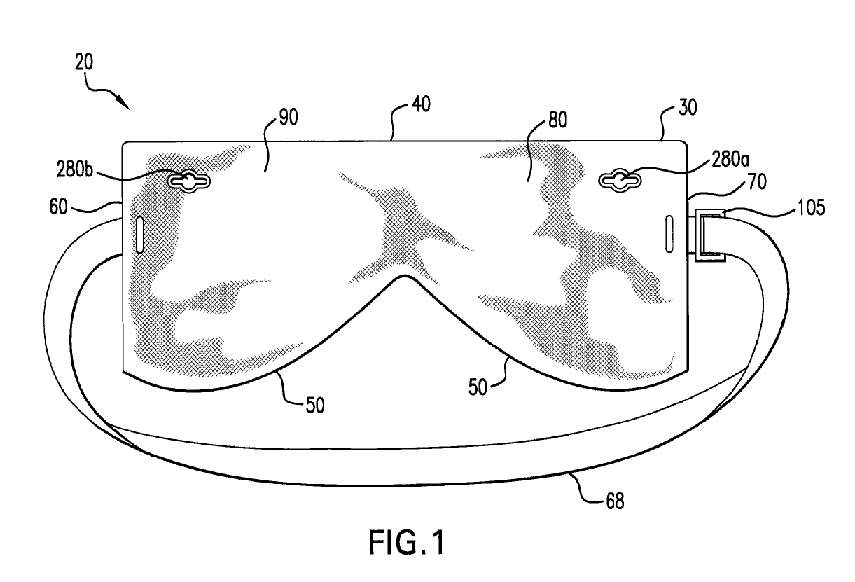

Referring to FIG. 1, in an embodiment, the present invention provides a compress assembly or system comprising a thermally adjustable pack 20 configured to be applied against the eye region of a user's face to apply a sufficient heat or cold source to the user's eye region to provide a therapeutic benefit to the user. By providing a therapeutic benefit to the user, the compress assembly improves a given user's condition compared to the same user's condition prior to use. Accordingly, pack 20 comprises a thermally activatable substance whose temperature can be regulated or adjusted by applying various degrees of heat or cold. Such a substance is capable, at a minimum, of being warmed or cooled so that it achieves a temperature that is substantially different from room temperature, and sustains the achieved temperature for a relatively long period of time and with a relatively slow period of decline back toward room temperature. By sustaining an achieved temperature for a relatively long period of time with a relatively slow period of decline back toward room temperature means that if 2.5 ounces of such a substance were heated from room temperature (72° F.) to a temperature of 135° F. and left in an uncovered condition (without insulation), the substance would maintain a temperature of at least 115° F. for at least five minutes after heating had taken place. Non-limiting examples of thermally activatable substances include water; various gelatinous materials such as solid or semi-solid gels; dried vegetables and cereals such as rice, beans, corn, and peas; water-containing food products such as potatoes and apples; and various other vegetables and food products. In a preferred embodiment, the thermally activatable substance is a gelatinous substance (also referred to herein as a “gel” or “gelatinous material”) and the thermally activatable pack is a gel pack. The below-described embodiments will be described with respect to a gelatinous substance although it is understood that other thermally activatable substances can also be used.

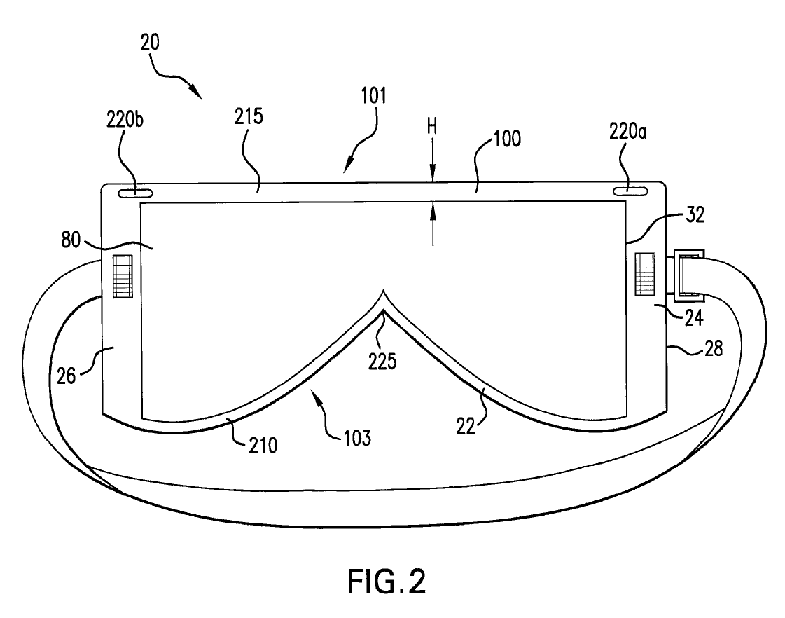

Referring again to FIG. 1, gel pack 20 comprises a casing 30 having a top portion 40, a bottom portion 50, a right portion 60, a left portion 70, a front side and a back side. As used herein in relation to the below description and accompanying figures, the terms “top,” “bottom,” “left,” “right,” “front,” and “back” refer to the orientation of the gel pack and compress assembly in relation to the user, in an applied position on the user's face when the user is standing upright (a position known in the art as the “anatomical position”) and facing out of the page toward the viewer. Of course, the gel pack and compress assembly can be used either in an upright (sitting or standing) or recumbent position. Reference to the user standing upright is only to provide a standard by which to understand the above-referenced locational terms. The front side of the gel pack is the side that faces outwardly and is the side illustrated in FIG. 1. The back side is the opposite side of the gel pack which faces the user in an applied position of the gel pack (i.e. when the gel pack is in use). Gel pack 20 defines a chamber 80 (illustrated more clearly in FIG. 2) holding a gelatinous thermal substance 90. In certain embodiments, casing 30 comprises at least two layers of flexible sheets sealed about their edges to form chamber 80. In those embodiments, casing 30 has a periphery defined by the sealed edges of the flexible sheets. The periphery of casing 30 can be co-extensive with the periphery of chamber 80 such that there is no space between chamber 80 and casing 30 as seen in FIG. 1. In other embodiments, as shown in FIG. 2, the periphery is divided into a top lip 101, which can further be divided into a top left lip 100 and a top right lip 215; a bottom lip 103, which can further be divided into a bottom left lip 22 and a bottom right lip 210; a left side lip 24; and a right side lip 26, where the lips are the regions between the outermost edge 28 of casing 30 and the outermost edge 32 of chamber 80 (and therefore such lips contain no gelatinous material).

In certain embodiments, top lip 101 has a height sufficient to accommodate fasteners to attach the gel pack to an external frame or to attach a gel pack to an external frame and a sheet(s) (as described in more detail below). In addition or alternatively, in certain embodiments, left and right lips 24 and 26 have a length sufficient to accommodate such fasteners. In other embodiments, the bottom lip 103 has a height sufficient to accommodate such fasteners. In other words, the periphery of the casing can be sized to accommodate fasteners in various different locations. With specific reference to the embodiment illustrated in FIG. 2, apertures 220, one defined by top left lip 100 and the other defined by top right lip 215 are shown that can receive fasteners, such as buttons, for example, to fasten the gel pack to an external frame or to an external frame and a sheet. Alternatively, the top lip 101 of the gel pack can have fasteners attached thereto to secure the gel pack to an external frame or to a sheet(s) and an external frame. In certain embodiments, top lip 101 has a height H of between about 2 millimeters (mm) and 20 mm. In a more preferred embodiment, top lip 101 has a height H of between about 10 mm and 15 mm. Of course, as shown in FIG. 1, gel pack 20 can be configured such that the outermost edge of top portion 40 of casing 30 is coextensive with the outermost edge of the top portion (not shown) of chamber 80 and can still accommodate fasteners. For example, the top portion 40 of gel pack 20 in FIG. 1 can define apertures 280 similar to the apertures of FIG. 2, as long as the edges of the apertures are sealed to prevent leakage of the gelatinous substance.

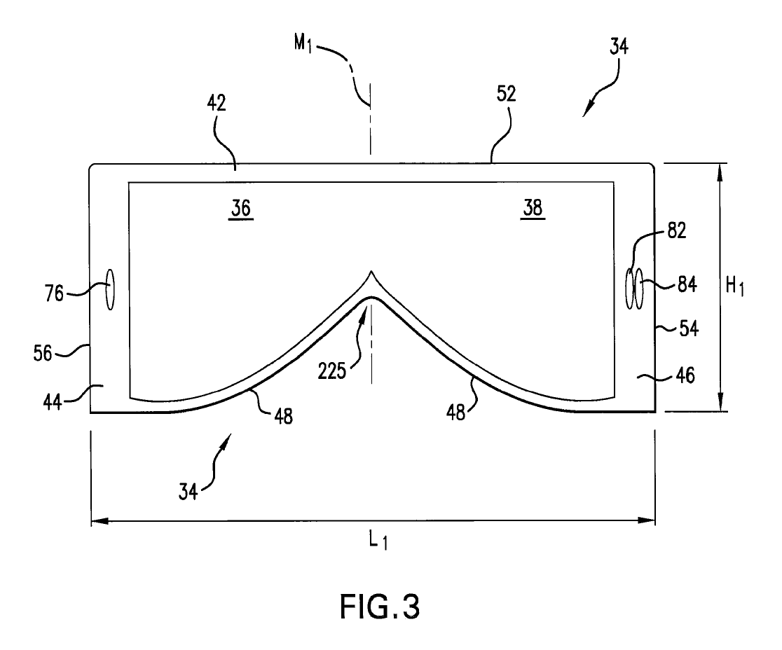

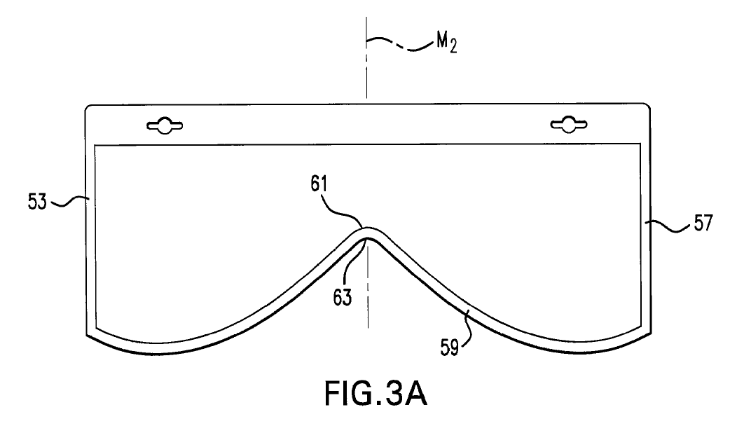

The gel pack can have various configurations. Such configurations can depend, for example, on the body region, such as the eye region, of the user that the gel pack is applied against. For example, referring to FIG. 3, a gel pack can be in the form of a mask 34 that is configured to cover the periocular and periorbital regions of the user's face. As shown in FIG. 3, the mask has a generally rectangular configuration with a substantially triangular notch 225 for the nasal area. Of course, the mask could have other configurations as well such as a generally oblong configuration with a similar cut out to receive the nasal wings. As shown in FIG. 3, the mask has a centerline M₁ dividing the mask 34 into a right section 36 and a left section 38 that spans over both the left and right eyes respectively of the user in an applied position. Specifically, right section 36 is configured to be applied against the right periocular and periorbital region of an average user's face and left section 38 is configured to be applied against the left periocular and periorbital regions of an average user's face. By being configured to be applied to certain eye regions of an average person's face, a gel pack is configured to cover these regions of an average person's face and provide therapeutic benefit to the user. However, while the gel pack is configured to cover certain regions of an average person's face, the gel pack can be used on a user who does not have average facial dimensions but who still can receive therapeutic benefit from a gel pack.

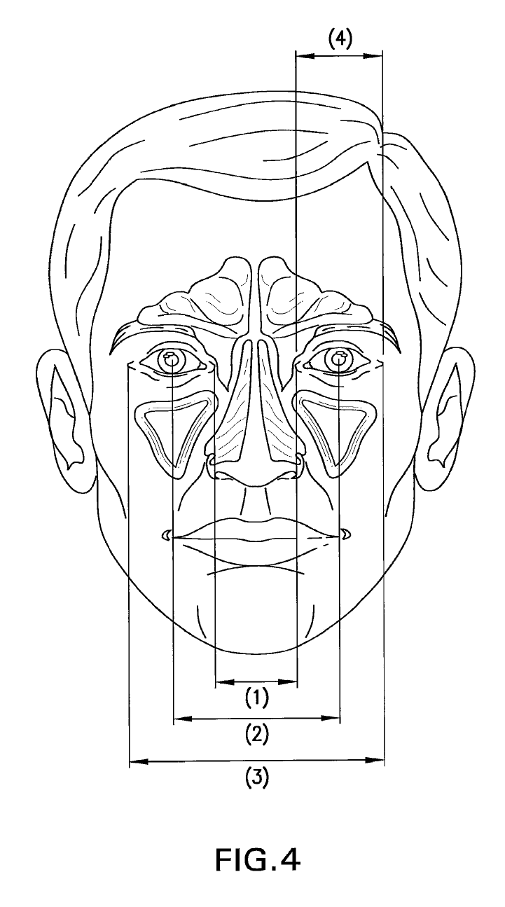

Referring to FIG. 4, which provides a schematic illustration of an “average” adult male face, certain periocular dimensions of the average human face, including children's faces, are as follows: the distance (1) between the inner canthus of the left eye and the inner canthus of the right eye (1) is about 20 to 36 mm; the distance between the center of the right pupil and the center of the left pupil (the interpupillary distance) (2) is about 46 to 75 mm; the distance between the outer canthus of the left eye and the outer canthus of the right eye (3) is about 71 to 105 mm; and the distance between the inner canthus of the left and right eye and the outer canthus of the respective left and right eye (the horizontal palpebral fissure width of each eye) (4) is about 25 to 32 mm. Of course, as mentioned above, other users' facial dimensions may fall slightly out of these ranges but the devices and methods will still be of therapeutic benefit to such users. The above-referenced measurements simply provide a guide to understand the general configuration of the gel packs when used for the eye regions.

Although the left and right sections of a gel pack can be separated from one another such that they are not in fluid communication, in the embodiment shown in FIG. 3, the left and right sections are in fluid communication with each other (i.e. there is no physical separator or divider between the two sections). This feature may be preferred and proved advantageous when the gel pack is activated in a microwave oven and where there is uneven heat distribution applied to the gel pack. Pressure can be applied to the unevenly heated gelatinous substance (i.e. applying back and forth pressure between the two sections of the gel pack) to allow redistribution of the gelatinous substance that resulted in a more homogenous heating effect when in use.

Regarding the specific configuration of a mask that can be used as a gel pack as illustratively shown in FIG. 3, mask 34 has a top portion 42, a right side portion 44, a left side portion 46, and a bottom portion 48. In the embodiment shown in FIG. 3, top portion 42, left side portion 46 and right side portion 44 have relatively straight edges, 52, 54, and 56 respectively but curved edges are also possible. Left and right side portions transition into a bottom portion 48 shaped like a bell curve which essentially creates a notch 225 to accept the nasal wings of the user. Alternatively, the peak of the notch 225 could be angled instead of curved as shown in FIG. 2. Still alternatively, as shown in FIG. 3A, the right lip 53 and the left lip 57 can transition into a bottom lip 59 shaped like a bell curve where both the top 61 of the bell curve shaped section of bottom lip 59 (which center line M₂ passes through) and the bottom 63 of the bell curve shaped section of bottom lip 59 (which center line M₂ also passes through) are both curved.

Referring back to FIG. 3, in certain embodiments, mask 34 has a maximum length L₁, of between about 4 inches and 11 inches. In a preferred embodiment, mask 34 has a maximum length L₁ of between about 5.75 inches and 9.0 inches.

Maximum length L₁ is taken by measuring the length of an imaginary line between the two farthest points on the left and right portions of the mask, the imaginary line being perpendicular to centerline M₁. In certain embodiments, mask 34 has a maximum height H₁ of between about 2 inches and 6 inches. In a preferred embodiment, mask 34 has a maximum height H₁ of between about 2.5 inches and 4.5 inches. Maximum height H₁ is taken by measuring the length of an imaginary line between the two farthest points on the top and bottom portions of the mask, the imaginary line being parallel to centerline M₁.

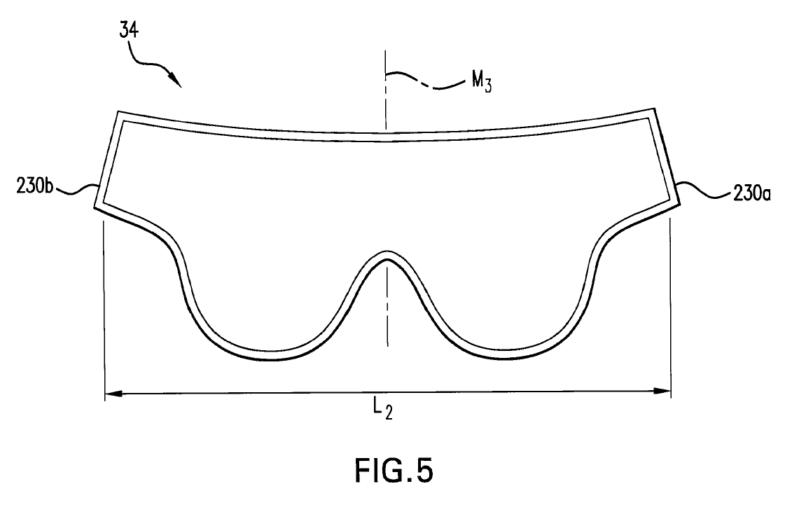

Referring to FIG. 5, in another embodiment, a gel pack is configured similar to the mask of FIG. 3 but with lateral wings 230a and 230b that extend past regions of the left and right side portions respectively of the mask illustrated in FIG. 2 and FIG. 3. Such a configuration may be particularly useful if it is desired to apply thermal compression to the periocular region and the periorbital region, including the temples of the forehead, for example. In other embodiments, the width of the mask could be reduced so that thermal compression is not applied to the entire periorbital region but to the periocular region and the temples of the forehead. In certain embodiments, the mask 34 in FIG. 5 has a maximum length L₂ of between about 6 and 12 inches. The maximum length L₂ of mask 240 is calculated in the same way as the maximum length L₁ of mask 34 is measured as described above with reference to FIG. 3.

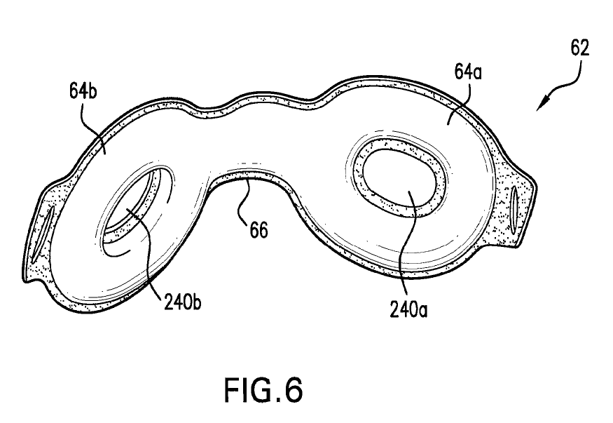

Referring to FIG. 6, in other embodiments, a gel pack can be in the form of goggles 62 which comprise two ring-shaped eyepieces 64 (a left and a right eyepiece) joined together by a longitudinal bridging member 66 that connects the two eyepieces. Bridging member 66 is adapted to bridge the nose of the user. Such an embodiment may be desired if only the immediate periorbital region of the user's face, and not the eyelids, is desired to be covered. As can be seen from FIG. 6, in this embodiment, eyepieces 64 define two apertures 240a and 240b which are substantially centrally located in the eyepieces and are sized to accommodate an average user's eyes. These apertures prevent any gelatinous material from being pressed against the eyeball of the user. In other embodiments, there are no apertures but it is preferable that the volume of gelatinous material near the center of the eyepieces is an amount that will not provide discomfort to globes of the user's eyes (i.e. the volume of gelatinous material in the center of each eye piece is less than the volume of gelatinous material in the area of the eyepiece surrounding the center). In certain embodiments, each eyepiece has a circumference of between about 9.0 inches and 12.5 inches. Of course other configurations of eyepieces 64 are also contemplated such as rectangular or oval, for example.

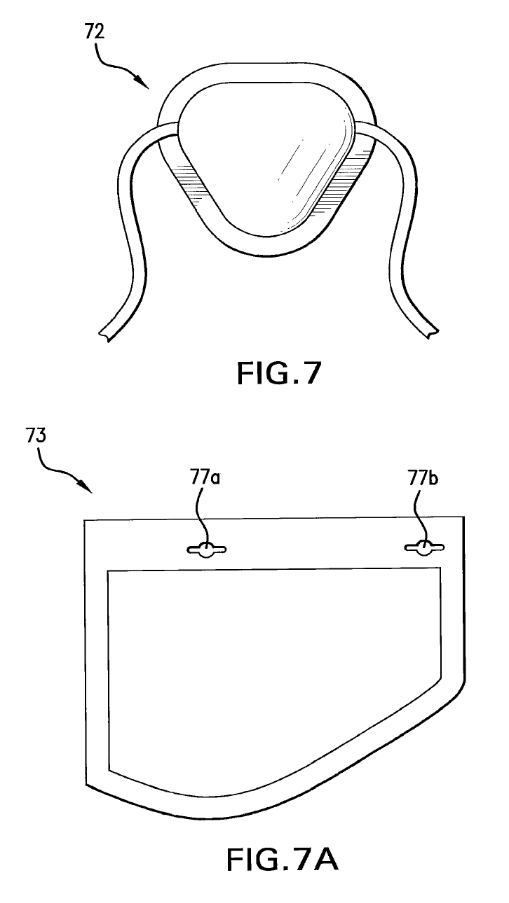

Referring to FIG. 7, in other embodiments, a gel pack is in the form of an eye patch 72 which is configured to cover only one eye region of the user (i.e. either the left or the right eye region). Such a configuration may be useful where therapy is desired for only one eye region. In the embodiment shown in FIG. 7, eye patch 72 has an oval shape but other shapes are also possible such as rectangular or circular, for example. The exemplary dimensions of eye patch 72 can be the same as a single eyepiece of goggles 62 (as shown in FIG. 6) particularly, but not exclusively, when it is desired for the gel pack to cover only one side of the periorbital region of the user's face. Alternatively, as shown in FIG. 7A, the exemplary configuration of eye patch 73 can be substantially similar to a single section of mask 34 as shown in FIG. 3, particularly, but not exclusively, if it is desired for the gel pack to cover only one side of the periocular and periorbital regions of the user's face. In the embodiment shown in FIG. 7A, the gel pack 73 defines apertures 77a and 77b on the top portion thereon to receive fasteners such as button or snaps, for example, to attach to an external frame or an external frame and a sheet(s) as described in more detail below. Of course, the top portion can include other types of fasteners to secure itself to the external frame.

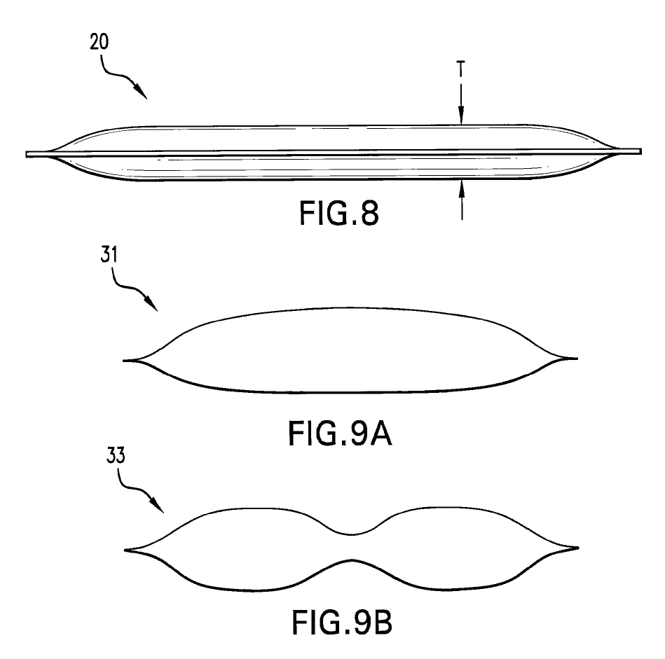

Referring to FIG. 8, in certain embodiments, gel pack 20 has a maximum thickness T between about 0.25 inches and 0.35 inches as taken from the center of the gel pack in a resting position when the gel pack has not been subject to manipulation and the gel is substantially evenly distributed throughout the pack. In certain embodiments, the maximum thickness T of the gel pack does not exceed 0.8 inches so that the gel pack does not have a bulging configuration in a resting position as gel pack 31 does as shown in FIG. 9A or a bulging configuration in an applied position as gel pack 33 does as shown in FIG. 9B. Thus, in a planar configuration, when the gel pack is applied against the eye region of the user, the casing achieves a relatively flat and deformable configuration when it contacts the desired eye regions (i.e. the periocular region or periorbital region of the face), rather than pressing with a bulgingly convex surface against the relative convexity of some of the user's anatomy (such as the globes of the eyes).

Because the gelatinous substance is slippery and difficult to control, a casing is used to contain the gelatinous substance so that the user does not come in contact with the gelatinous substance. The casing can be fabricated from any suitable material to hold the gelatinous substance and to allow thermal diffusion (that is, ready conductivity of heat or cold to the skin, when the gel pack is placed directly or indirectly against the skin). Preferably, the casing of the gel pack is fabricated from any suitable material that can withstand repeated exposure to heat and cool with minimal deformation and without significant degradation. By “minimal deformation” is meant that the gel pack maintains a configuration after 100 heating cycles (with exposure to temperatures between about 100° F. to 160° F.) and/or cooling cycles (with exposure to temperatures between about 40° F. to 0° F.) that is similar enough to its configuration before first use such that it can still perform its intended function and provide therapeutic benefit to the user. By “significant degradation” is meant that the casing degrades to the point that it can no longer perform its intended function and provide therapeutic benefit to a user after 100 heating cycles and/or cooling cycles (at the range of temperatures indicated above).

A preferred material is one that is also flexible enough such that it can sufficiently conform to and be in direct contact with the desired eye regions of the user. The material should also preferably be resistant to any negative chemical effects of the gelatinous substance. Preferably, the material of the casing is waterproof to protect the casing from exposure to moisture (such as in the case of the gel pack being used in conjunction with moistened sheets as described in more detail below). To reduce the chance of microbial buildup with repeated use, materials that can be cleaned with soap and water or alcohol pads are preferred.

Non-limiting examples of materials for the casing including thermoplastic polymers films such as polyamides, polyolefins (including polyethylenes and polypropylenes) and suitable combinations thereof. Films containing polyethylene may confer greater temperature conductivity than polyvinylchloride and vinyl materials and are therefore preferred. Further, vinyl materials have been reported to undergo some degradation with release of toxic chemicals when microwaved. Therefore, in a preferred embodiment, the casing is not fabricated from a vinyl material.

Preferably, the periphery of the casing is fabricated from a material of sufficient strength to resist rupture under normal use. However, it may be preferable to allow such rupture in a controlled manner when the chamber pressure is raised to a dangerously high level, such as when a gel pack is inadvertently microwaved for an excessively long time. In such circumstances, slow leakage of contents through a deliberately-ruptured seal would be preferable to explosion of the gel pack. To allow for this slow leakage of contents, the final heat-sealing of the periphery of the casing following gel insertion can be adjusted such that the final heat seal is weaker and less able to withstand an increased internal pressure (as from expansion of water vapor volume of the gel pack chamber during heating) than the material of the casing itself.

As described above, a gel pack includes a chamber that holds a gelatinous substance. The gelatinous thermal substance has characteristics that allow it be malleable enough to conform to the external contour of the user's eye region and to act as an effective thermal reservoir. Specifically, the gelatinous substance preferably comprises a readily deformable gel that can be repeatedly heated and cooled (including freezing) with no appreciable decrease in performance over time. Such heating includes microwaving the gelatinous substance or exposing the gelatinous substance to hot water at temperatures ranging from about 100° F. to 212° F. Such cooling includes placing the gel pack on ice (for example in an ice bath) or within a source of cold air such as a freezer or refrigerator at temperatures ranging from about 40° F. to 0° F. Further, the gelatinous substance preferably comprises a gel that can maintain a desired range of viscosities when subjected to the range of temperatures a user may select. Some stiffening of the gelatinous material would be expected at very low temperatures and some softening at very higher temperatures but the parameters of the gelatinous substance should be preferably such that the substance remains malleable enough so that the user can manipulate the gelatinous substance to maintain its position in a specific eye region for at least 5 minutes. In preferred embodiments, the gelatinous substance can be heated or cooled at least 100 times (at heating temperatures ranging from about 100° F. to 160° F. and cooling temperatures ranging from about 60° F. to 40° F.) while still maintaining its intended function and providing therapeutic benefit to the user.

Parameters of the gelatinous substance that allow for the maintenance of such intended functions include, for example, the composition of the gelatinous substance, the volume of the gelatinous substance, the surface area of the casing, and/or the viscosity of the gelatinous substance. Regarding the composition of the gelatinous substance, non-limiting examples of gelatinous substances include the gelation of xanthan gum, locust bean gum, gum tragacanth, and guar gum; hydroxypropyl cellulose, absorbent and superabsorbent polymers including CARBOPOL™, carboxymethyl cellulose, sodium polyacrylate; similar materials; and suitable combinations thereof.

Regarding the volume of the gelatinous substance, the volume of the gel should provide a sufficiently large mass to serve as an effective thermal reservoir yet not cause the gel pack to bulge and transmit excessive pressure on the eyeball. Of course, in part, the volume of the gelatinous substance depends on the surface area of the casing. For example, if the surface area of the chamber (which is the portion of the gel pack excluding the lips in embodiments where the casing has a peripheral lip as shown, for example, in FIG. 3A) is between 17 to 18 square inches, the volume of the gel is preferably between 2 to 4 ounces. Alternately, if the surface area of the chamber is between 21 and 22.5 square inches, the volume of gel is preferably between 4 to 5.5 ounces. Of course, other dimensions and gel volumes could be used.

The relationship between the amount of gel and the volume of the chamber within the gel pack could be modified to produce gel packs of different sizes and weights, and with different surface characteristics. Gel packs in which the ratio of gel to chamber volume is relatively low would tend to produce packs in which there is relatively little bulging of the surface, and therefore little pressure against the globes of the eyes, but in which the thermal effect of the gel pack is somewhat limited in duration owing to the relatively low volume of gel. Conversely, gel packs in which the ratio of gel to chamber volume is relatively high would tend to produce packs in which there is somewhat more of a bulging contour, and hence somewhat more pressure against the globes of the eyes, but in which there is a more lasting thermal effect owing to the larger volume of gel.

It is desirable that any combination of mass and distribution of gel be sufficient to provide adequate treatment as a thermal compress for a standard duration of treatment. As an example, not meant to be limiting, when the surface area of the chamber is 17.5 square inches and the gel mass is 2.5 ounces, once the gel is heated to 140° F. under experimental conditions, the gel pack is of sufficient mass and has characteristics sufficient to provide a sustained thermal effect for five minutes, such that at the end of five minutes at room temperature, the temperature of the gel pack remains above 110° F.

Of course, the above-described volume and surface areas are only exemplary and the volume and surface area of the respective gelatinous material and casing can be controlled for other configurations of the gel pack in order to achieve a relatively flatter and less bulging contour with a lower gel weight, or a relatively more bulging contour with a higher gel weight.

In the particular instance in which the user desires to employ a gel pack with a larger volume of gel, the user may find it useful to adjust the body part so that gravity pulls the gel pack away from, rather than toward, the body part. In the example of an eye compress, if the user tilts his or her head forward, the gel pack will be positioned away from the eyes, and would therefore spare the user excess pressure on the eyeballs. Doing so would allow the user to employ a heavier gel pack, with a longer thermal effect, without ocular discomfort.

Thus, one way of increasing the duration of thermal treatment is to increase the volume of gel within the gel pack, either by using more gel in a pack of a given volume, and/or by increasing the volume of the pack.

Another possible way of increasing the duration of thermal treatment is by using two or more lower-volume and more planar gel packs, one stacked behind the other. Using lower-volume gel packs in this manner would tend to lower manufacturing, distribution, and sales costs (since only one size and shape of gel pack would be produced), and would tend to give users greater choice during each treatment (allowing each user to select, during a given treatment, whether to use one or more gel packs). This method of stacking gel packs is not previously mentioned in the art, presumably because the challenge of providing a gel pack that is of sufficient volume to sustain a desired hot or cold temperature range, but is also of minimal enough volume that it will not place undue pressure on a sensitive body part such as the eyes, has not previously been effectively addressed.

In general, a lower gel volume-to-chamber area ratio will allow more manipulation of the gelatinous substance so that the user can manipulate the gelatinous substance more freely, and press it into better conformation against his or her own anatomy by creating small bulges in one location and small depressions in another. Preferably the viscosity and volume of the gelatinous substance is such that it allows the user to tailor the amount of gelatinous substance applied against certain eye regions. For example, if the user prefers that a greater thermal effect be applied against a portion of the maxillary sinus, then the user can shape the gelatinous substance such that the gel is easily pressed inward against the region of the face overlying the maxillary sinus. In addition, once deformed to its new inward configuration, the gelatinous substance can be stiff enough so that it will tend to hold its shape for at least 5 minutes relative to this new configuration and will not flow back to its previous configuration by force of gravity.

Ideally and in a preferred embodiment, the material of the casing of the gel pack will work in a synergistic manner with the gelatinous substance contained within it in order to produce a desired outcome of conformation to the user's body part. For example, once the gel is deformed to a desired shape by the user, the inherent stiffness and shape of the casing of the gel pack should preferentially support the shape of the gel in its desired configuration, and help it to resist flowing downward by force of gravity.

Preferably the gelatinous substance has a high water content, allowing rapid energizing by microwave radiation as well as prolonged heat retention due to water's high specific heat. Preferably, the gelatinous substance is biocompatible and non-toxic although it is not expected that a user would come into direct contact with the gelatinous substance during use. Additives could be used to raise the boiling point of the gelatinous substance thereby reducing the risk of vapor production and gas expansion during heating, which could, with prolonged microwave heating, cause the gel pack to burst. Non-limiting examples of such additives include polyethylene glycol. Other additives can also reduce the freezing point, allowing the gelatinous substance to attain low temperatures while maintaining softness and deformability. Non-limiting examples of such additives include sodium chloride. The gelatinous substance could be prepared and sealed in the pack under vacuum conditions in order to minimize the presence of air, thereby further reducing the risk of gas expansion during heating. Lowering the presence of air or gas could also allow for more uniform heating of the gel.

Referring back to FIG. 1, in certain embodiments, a compress assembly of the present invention further comprises a strap 68 attached to casing 30 to secure gel pack 20 against the body region of the user, (which in the embodiment shown in FIG. 1 is the eye region) and to exert a compressive force to the gel pack. The strap can be made of any material sufficient to perform these functions. For example, the strap can be fabricated from an elastic stretchable material. Alternatively, the strap can be a non-stretchable material such as a string or ribbon which can be tied to secure the gel pack to the user's face. Preferably, the strap is adjustable allowing the user to exert variable degrees of compressive force to the gel pack. For example, an elastic strap can include a buckle 105 to adjust the tension of the strap according to not only the circumference of the user's head but also according to the degree of compression desired to be applied against the user's eye region. In embodiments where the strap is non-elastic (such as, for example, a string or ribbon), the strap can be tightened by pulling on the ends of the strap to control the compressive function of the strap. Of course, other materials and configurations of strap 68 could also be used. Referring to FIG. 2, the strap can be attached to the left side lip 24 and the right side lip 26 of casing 30. Of course strap 68 can be attached to other portions of casing 30 so long as strap 68 performs its intended function. In FIG. 2, strap 68 is threaded through slit 76 (illustrated in FIG. 3) of casing 30 and secured to gel pack 20 via an interference fit with slits 82 and 84 of casing 30 (again illustrated in FIG. 3). However, other means of attaching strap 68 could also be used. For example, the strap could be glued or stitched onto casing 30. Alternatively, the strap can be integral with the casing such that the casing and strap are made from the same material and are one-piece in the sense that the strap is not separable from the casing using a normal amount of force without damaging the integrity (i.e. tearing) either the strap and/or the casing. Therefore, strap 68 can be removably or permanently affixed to the casing. Non-limiting examples of material from which strap 68 can be fabricated from include fabrics, plastics, woven elastics, and certain pliable elastic polymers.

A compress assembly and system of the present invention further comprises an external frame actively or passively positioned against the front side of a gel pack. Specifically, the external frame is attachable to or otherwise positionable against the outwardly facing side of the gel pack (i.e. the side of the gel pack that will not be in contact with the patient's body region in an applied position of the gel pack). Specifically, the external frame is passively positionable on the outwardly facing surface of the gel pack to form a body compress system or actively positionable on the outwardly facing surface of the gel pack to form a body compress assembly. By being passively positionable against the gel pack, the external frame is positioned against the gel pack without the use of any mechanical means to attach the external frame to the gel pack in an applied position of the system. However, the external frame could be in communication with the gel pack via frictional engagement. By being actively positionable against the gel pack, the external frame is positioned against the gel pack via the use of mechanical means to attach the external frame to the gel pack in an applied position of the assembly.

In the former embodiment, the external frame includes a strap to compress the gel pack against the user's anatomy and provides a relatively firm base surface (as compared to a flexible surface such as is supplied by an elastic or soft woven fabric) that is designed to provide a source of external pressure against the gel pack directly over specific regions of the anatomy of the user. The user can then adjust the pressure of the external frame in order to optimize pressure against the body part. Thus, for example, in a body compress system designed for use on the eye region in which a gel pack is positioned against the eye region with the user in an upright position and in which the gel pack does not have a source of vertical support for its weight, an external frame that is passively positionable against a gel pack could be shaped to correlate with the general outline of the ocular anatomy (or to some portion thereof) in such a way that any increased pressure on the external frame would transmit pressure preferentially to the ocular anatomy (or to some portion thereof). During use, the stiffness of the external frame would provide some support to keep the gel pack in position against the body part. The stiffness of the external frame would also allow the user to position the frame in such a way that the lower edge of the frame rested on a body part inferior to the body part being treated, such that the bottom of the gel pack was supported on the junction between the bottom edge of the external frame and the user's body. This would then allow the user to select from a variety of compression tensions in a strap that tightened the external frame against the gel pack. The stiffness of the external frame would also make it easier for the user to determine and adjust the exact location in which this pressure was to be exerted, simply by pressing the relatively stiff frame element with his or her hands.

If the external frame in a body compress system designed for use on the eye region defined relief apertures corresponding to the region of the eyes themselves, the presence of such relief apertures would selectively decrease the direct pressure of the external frame against the eyes, and would also allow the user to directly manipulate the gel pack by providing direct access through the relief apertures.

In embodiments where the external frame is actively positionable against the gel pack, the gel pack or the external frame includes a strap and the external frame is fastened to the gel pack to support at least a portion of the gravitational weight of the gel pack so that in an applied position, the gel pack does not sag to such a degree that the gel pack in no longer able to provide therapeutic benefit to the user. Such sagging can take place both in terms of the position of the entire gel pack as well as in terms of the gelatinous material within the gel pack.

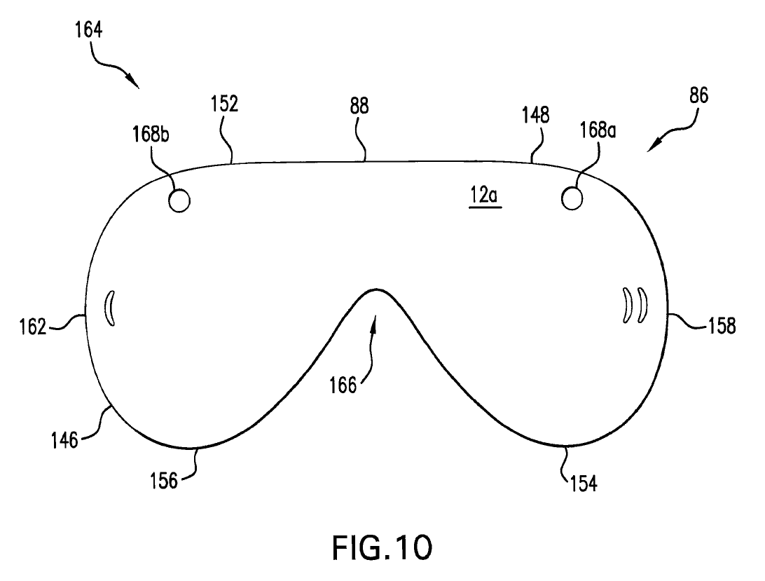

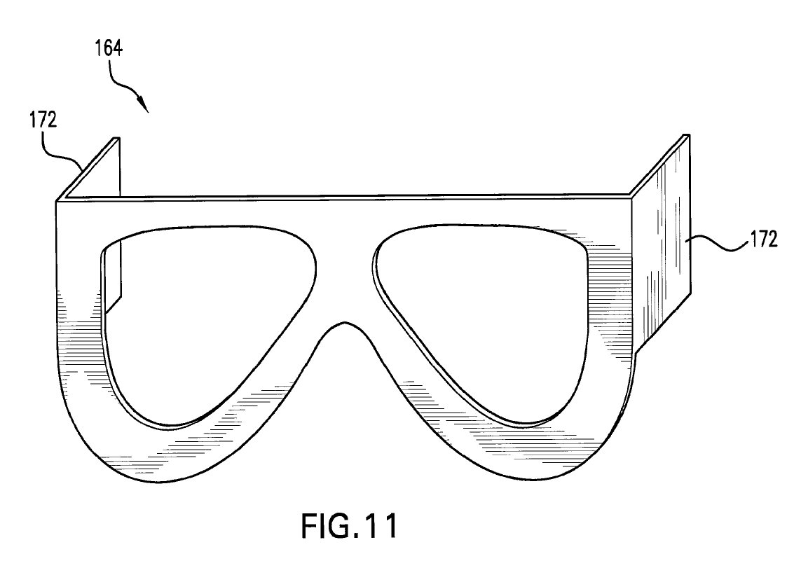

The gel pack used in conjunction with the external frame can be configured as described above or can have a different configuration. Exemplary external frames are shown in FIGS. 10, 11, and 14. Referring to FIG. 10, an exemplary external frame 164 comprises a frame body 146 having a top left portion 148, a top right portion 152, a bottom left portion 154, a bottom right portion 156, a left side portion 158 and a right side portion 162. In the embodiment shown in FIG. 10, all the aforementioned portions of the frame body are curved but some or all of the portions can have straight edges as does the gel pack of FIG. 2. In the embodiment shown in FIG. 10, the right and left side portions transition into a bottom portion shaped like a bell curve which essentially creates a curved cut out 166 to accept the nasal wings of the user. Alternatively, the peak of the cut out could be angled instead of curved (similar to the notch 225 of gel pack 20 shown in FIG. 2). Preferably, an external frame mimics the outline of the gel pack that is used with the external frame as part of the eye compress assembly or system. For example, if the gel pack is configured as shown in FIG. 5, external frame 164 can also be similarly figured, as shown in FIG. 11 with lateral wings 172 such that, in use, external frame 164 applies compression to the temples. Of course, an external frame could be configured to have lateral wings that cover the temples of the head during use irrespective of the configuration of the gel pack. In any event, preferably, an external frame is sized and shaped such that the gel pack extends beyond the edges of the external frame on all sides so that the user's face is contacted in all applied areas by the gel pack rather than directly by the external frame.

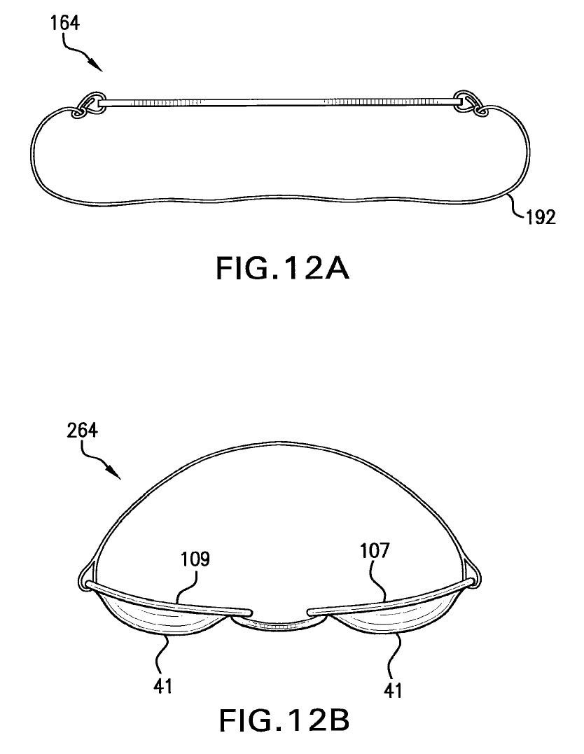

In certain embodiments, the external frame assumes a generally flat or planar conformation when in a resting position. As used herein, a “resting position” refers to the position of the external frame when it is not applied against the body region of the user (i.e. an applied position) and is resting on a flat surface. This resting position of an external frame can be seen best in FIG. 12A, which is a plan view of the external frame 164 of FIG. 10 (but also includes a strap 192) This generally flat or planar configuration is in contrast to the configuration of external frame 264 in FIG. 12B, which has a convex outer surface 41 for the section 107 that covers the right eye region and the section 109 that covers the left eye region (which is the surface that would face away from the user's face in an applied position). Thus in embodiments where the external frame has a generally planar configuration, when the eye compress assembly is applied against the eye region, the external frame has the ability to press the gel pack directly against the desired eye regions (i.e. the periocular and/or periorbital region of the face), which would not occur if the external frame had a convex configuration in a resting position.

As mentioned above, the external frame can be actively or passively positioned against the gel pack. With respect to being actively positioned against the gel pack, the external frame can be physically attached to the gel pack to support at least a portion of gravitational weight of the gel pack such that the gel pack does not sag on the user's face to such a degree that the gel pack no longer is able to provide therapeutic benefit. In such an embodiment, the external frame is fabricated from a material stiff enough to support at least a portion of the weight of the gel pack such that the external frame does not buckle when in a vertical position and when the gel pack is attached to the external frame and secured against the eye region of the user. An external frame can be attached to the gel pack in any suitable way. For example, the external frame can be permanently or removably attached to the gel pack in use. Regarding the former, an external frame could be glued or heat molded onto the gel pack during manufacture. Other means of permanently attaching an external frame to the gel pack are also possible. If an external frame is permanently attached to the gel pack, the external frame is fabricated from a material that is heat and cold resistant such that the external frame can be exposed to a heat or cold source without degrading to the point of losing its intended functions.

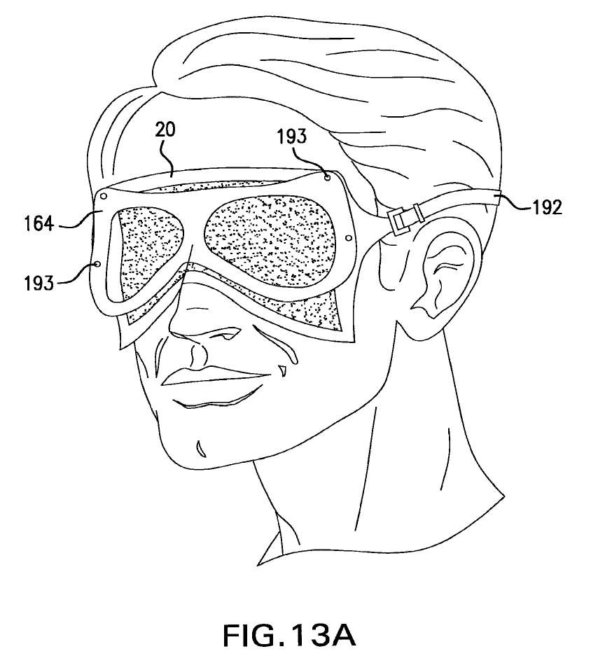

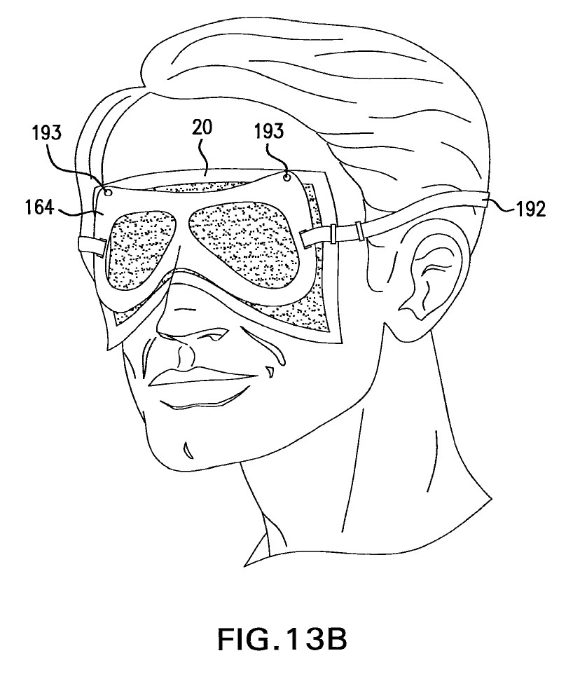

Regarding an external frame being removably attached to the gel pack in use, the frame can accommodate at least one fastener to secure the gel pack to the external frame. For example, as illustrated in FIG. 10, the external frame can define apertures 168a and 168b in top portion 148 and 152, respectively, of frame body 146 that are configured to receive buttons, string, snaps or other fasteners to attach to the top portion of the gel pack, in which case the external frame supports substantially all of the gravitational weight of the gel pack. Although FIG. 10 illustrates two apertures, an external frame can include more than two apertures in the top portion of the frame body. An external frame could also define any suitable number of apertures in bottom right and left portions 156 and 154 respectively and/or right and left side portions 162 and 158, respectively. Alternatively, external frame 164 could define a single aperture centrally located between top right portion 152 and top left portion 148. Still alternatively, the external frame could define at least one aperture on left side portion 162 and at least one aperture on right side portion 158 of frame body 146. In such an embodiment, the frame body supports at least a portion of the gravitational weight of the gel pack (but not as much as would be supported if the gel pack were attached to the top portion of the frame body) when in use. However, the gravitational weight that is supported in such an embodiment is enough to prevent the gel pack from sagging during use in such a way that it interferes with the therapeutic benefit of the device. The exact number and location of the apertures can vary so long as an external frame supports enough of the gravitational weight of the gel pack such that the gel pack does not sag in an applied position. Notwithstanding the exact number and location of apertures in external frame 164, the gel pack would have similar apertures, for example, as described above such as with respect to FIG. 2 in such embodiments. It is understood that if a strap 192 is attached to gel pack 20 then the top portion and at least one of the side portions or the bottom portion of the external frame accommodates fasteners 193 as shown in FIG. 13A. Alternatively, the side portions and the bottom portions of the external frame accommodate fasteners. If strap 192 is attached to external frame 164, then the top portion or the side portions of the external frame can accommodate fasteners 193 (in addition, if desired, to other portions of the external frame) as shown in FIG. 13B.

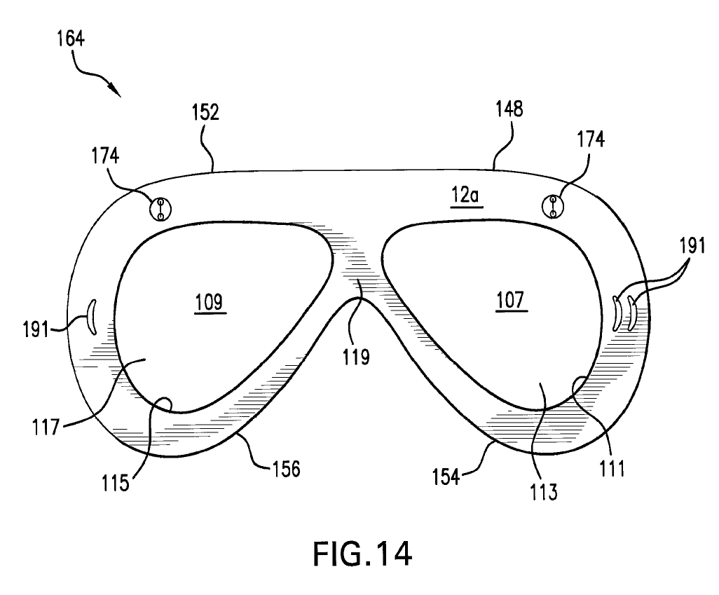

In addition to being designed to receive separate fasteners that are applied to the frame body, the frame body can contain fasteners that are already attached or attachable to the frame body. For example, the frame body can accommodate a magnetic strip to attach to a magnetic strip or metal strip disposed on a gel pack. In certain embodiments, as shown in FIG. 14, external frame 164 can have buttons or other fasteners already attached to the frame body. Such fasteners can be die-cut or molded, for example, into the frame body of the external frame. For example, FIG. 14 shows buttons 174 attached to the frame body of external frame 164. Of course the aforementioned fasteners are only exemplary and other fasteners can also be used so long as they achieve the function of attaching a gel pack and optionally a sheet (as described in more detail below) to an external frame. Non-limiting examples of other types of fasteners include velcro, clips, and male/female fasteners. Further, any number of fasteners can be used to secure the components of the eye compress assembly. In preferred embodiments, the fasteners are attached or attachable to the top portion of the frame body although the fasteners can be positioned at different locations on the frame body as described above with respect to apertures defined by the frame body to receive fasteners.

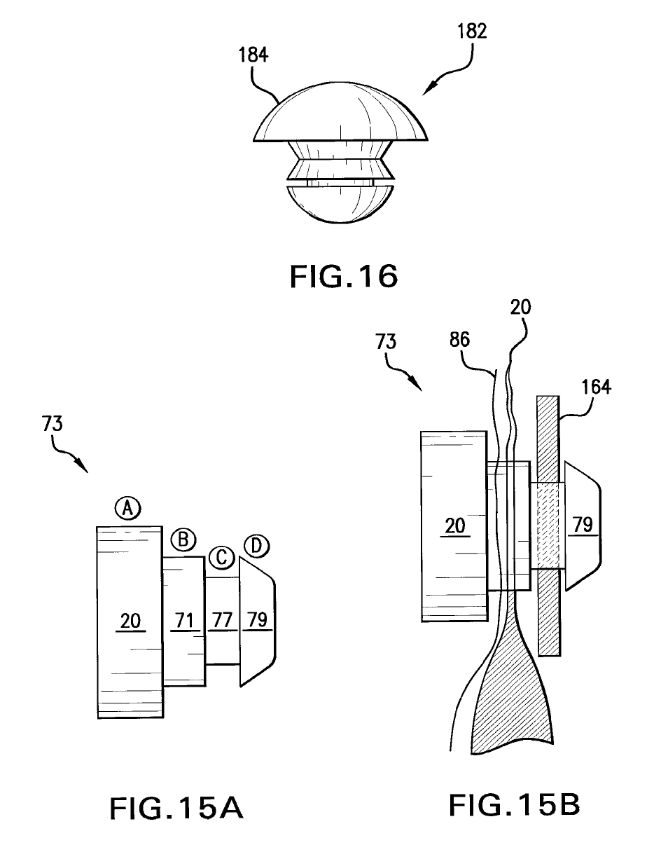

With specific reference to fasteners that are buttons, button-shaped fasteners may allow a broad range of users to intuitively understand the removable nature of the gel pack and an optional sheet. Referring to FIGS. 15A and 15B, in certain embodiments, a fastener 73 comprises a button 20 and a stepped down support shaft 71 connected to or integral with button 20 and sized to allow room for the insertion of both the top portions of the gel pack 20 and a sheet 86 as shown in FIG. 15B. Fastener 73 further comprises a post 77 that passes through the external frame 164 as shown in FIG. 15B and a stepped up anchor 79 that is on the opposite side of the frame as button 20 and that keeps button 20 locked to the external frame 164. Such buttons could be made of a variety of materials such as plastic or rubber. Buttons made of a soft silicone material were found to impart a flexible tension that allowed them safely and snugly to grip, rather than just passively support, the wet sheets used with the device and are therefore preferred (although not required). In one embodiment, shown in FIG. 16, a button 182 is designed and configured with a domed surface 184, rather than in a standard shape, in order to improve the ease with which users insert moistened sheets onto the buttons.

An eye compress assembly that includes an external frame can also include a strap 192 as shown in FIG. 13B. The strap can be attached to the gel pack or the external frame but in the embodiment shown in FIG. 13B, strap 192 is attached to the left and right portions of the external frame 164. In this embodiment, the strap is adjustable (although it is not required to be in other embodiments) such that tightening or loosening of the strap exerts a controllable horizontal pressure on the external frame. In certain embodiments, where the external frame is not attached to the gel pack via fasteners but is rather passively positioned against the user as shown in FIG. 18 via a strap, the external frame serves the function of applying compressive force against the user's anatomy.

However, in certain embodiments, where an external frame and a gel pack are attached together via at least their top portions as shown in FIG. 13B, the horizontal pressure exerted by the external frame strap is largely independent from the vertical support provided by the external frame to the gel pack via the one or more fasteners at the top portion of the eye compress assembly. As shown in FIG. 14, to achieve this effect, in certain embodiments, an external frame can include slits 191 in the right and left side of the external frame to accommodate a strap (such accommodation being similar to that described above with respect to a gel pack). In fact, in certain embodiments, both the gel pack and the external frame have slits in alignment with each other such that a strap could be threaded through both the gel pack and external frame for added securement of the gel pack to the external frame. Alternatively, a strap can be attached to an external frame via other exemplary means such as by being stitched or glued onto the external frame (again, such exemplary means being similar to that described above with respect to a gel pack). Therefore, the strap can be permanently or removably attached to the external frame. The strap can be made of a variety of stretchable or non-stretchable materials that preferably will not be either adversely affected by low levels of heat or heated by low levels of microwave irradiation such as the amount required to heat the gel pack, including fabrics, plastics, woven elastics, and some pliable elastic polymers. Preferably, the strap is able to be loosened sufficiently so that the external frame can serve simply to support the gel pack and optional soft sheets in relation to the body part, without compressing them against the body part. In general, the description of a strap as described above with respect to a gel pack applies to a strap attached to an external frame instead.

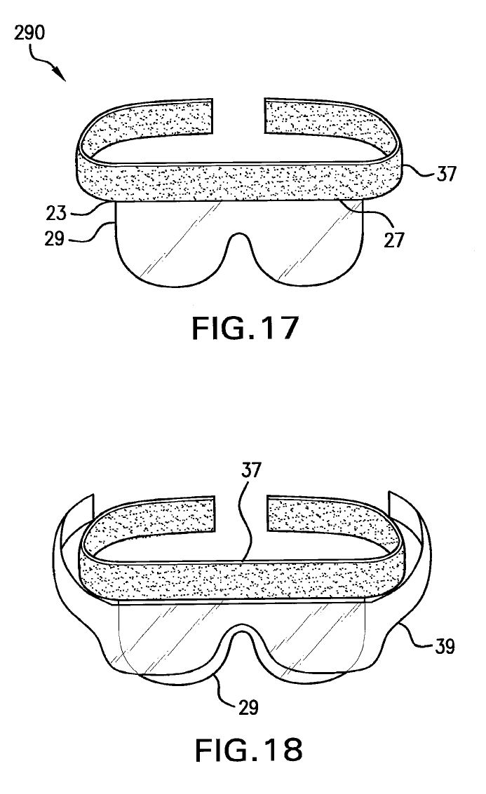

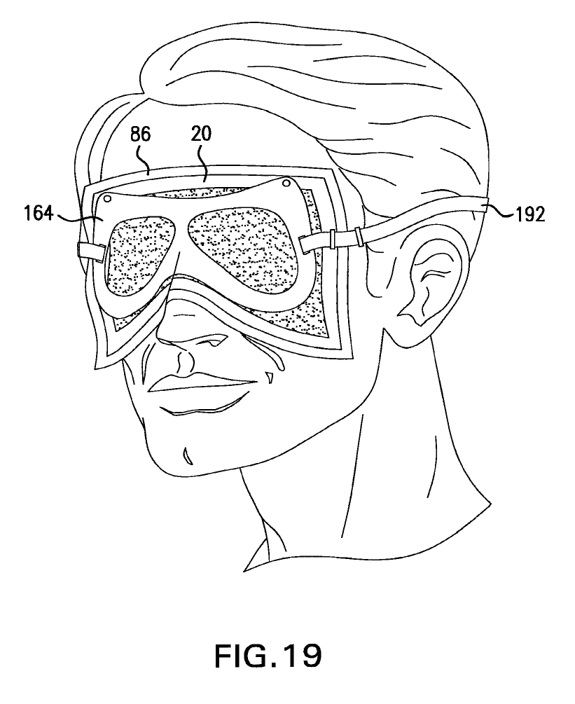

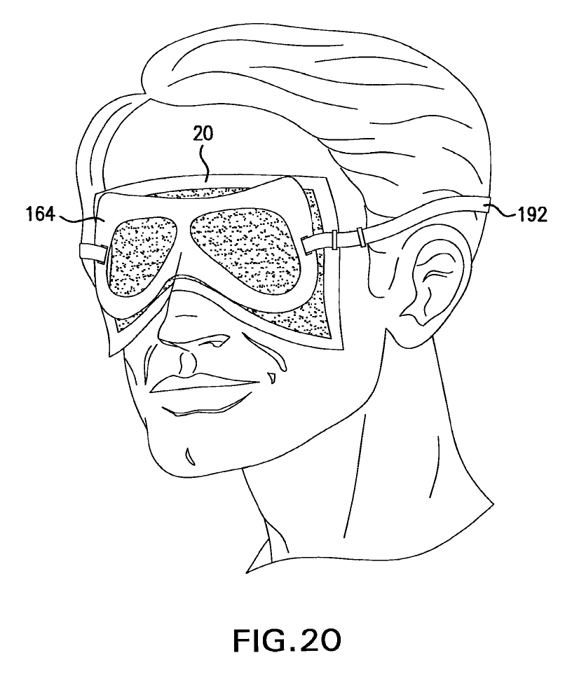

In certain embodiments, the external frame provides both a vertical support for the gel pack and a horizontal compression against the gel pack as shown in FIG. 13B. In another embodiment, the vertical support of the gel pack is supplied by one device and the horizontal compression against the gel pack is supplied by another device. For example, referring to FIG. 17, in an embodiment, a gel pack 290 is supported along its top edge (in an applied position) by a strap 37, whose bottom edge 23 is attached to the top edge 27 of gel pack 29 such that gel pack 29 depends from strap 37 and is vertically supported by strap 37, when strap 37 is wrapped about the body (e.g. the head in the case of an ocular compress). Specifically, in an applied position of the compress, the strap wraps around the body part for which treatment is being applied. The strap vertically supports the weight of the gel pack and holds it in position in relation to the body part but without compressing the gel pack against the body part to be treated. The strap can either be an integrated part of the gel pack or a separate component and can be removable or permanently attached to the gel pack. By being integrated, the strap is not removable from the gel pack without disrupting (i.e. tearing) the integrity of the strap and/or the gel pack when the strap is attempted to be removed using a normal, reasonable amount of force. By being a separate component, the strap can be removed from the gel pack without disrupting (i.e. tearing) the integrity of the strap and/or the gel pack when the strap is attempted to be removed using a normal, reasonable amount of force. If the strap is an integrated part of the gel pack, it can be made from the same material as the gel pack casing, and preferentially would not contain gel although in certain embodiments it could contain a gelatinous material. The strap could also be separate from the gel pack and could attach to the top edge of the gel pack in an applied position of the compress using a variety of attachment means known in the art. Once the gel pack is attached along its top edge to the bottom edge of the strap, the vertical weight of the gel pack is supported.

Once the vertical weight of the gel pack is supported by either an integrated or separate strap, a separate compression device, an external frame 39 can be applied to the outer surface of gel pack 29, as shown in FIG. 18 in order to allow the user to supply variable degrees of compression of gel pack 29 against the body part being treated. As seen in FIG. 18, the external frame, in use, wraps around the gel pack and the relevant body part (e.g. the head in the case of an ocular compress). In some embodiments, the external frame can be made of a stiff material, possibly similar to material described below. However, in other embodiments, the external frame is made of a soft, elastic or flexible material that is not stiff. The softness and flexibility of the external frame in such embodiments can allow the user to choose from a wider range of compression tensions in order to select the desired degree of pressure of the gel pack against the body part to be treated.

Referring back to FIG. 14, in certain embodiments, external frame 164 further comprises a bridging portion 119 that bridges the top portion (both left and right top portions 148 and 152) and the bottom portion (both left and right bottom portions 154 and 156). In this embodiment, external frame comprises a left section 107 whose internal periphery 111 defines a left relief opening 113 and a right section 109 whose internal periphery 115 defines a right relief opening 117. The relief openings are sized to allow a user to directly manipulate the position of the gelatinous substance in the gel mask by applying topical pressure to the gelatinous substance, a feature that is useful when the overall compressive tension in the frame is kept low but the user wants to selectively increase the compress effect in certain areas. Such relief openings also reduce some of the compressive pressure that would come from increasing the backwards tension on the frame in embodiments where a strap is attached to the frame. For instance, the relief openings allow the frame to focus such pressure on the periphery of the targeted body area rather than on the center of that area. The relief openings can have any suitable shape such as tear-shaped or circular, for example.

The relief openings can be directly exposed to the atmosphere or can be covered with a thin layer of fabric, plastic, foil, or other material which would cover the gel pack underlying the openings but would be flexible enough to allow the user easily to manipulate the gel. Certain materials could be selected to insulate the gel pack by reducing the amount of convective heat exchange with the surrounding air.

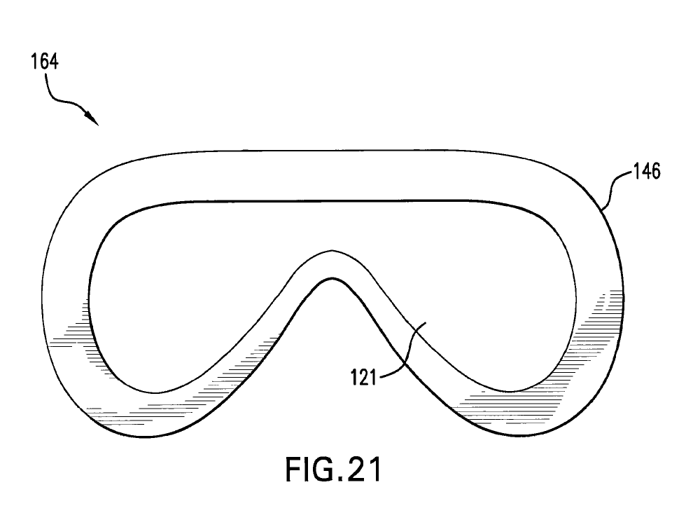

Referring to FIG. 21, in certain embodiments, the internal periphery of frame body 146 defines a single relief opening 121 which serves the same purposes as the dual relief openings described above with respect to FIG. 14.

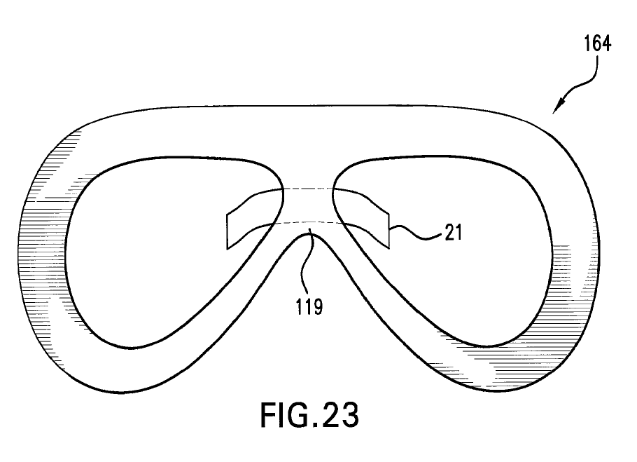

In reference to FIG. 23, in certain embodiments, an eye compress assembly further comprises a gel impressor 21, which is a flexible bendable strip that can be placed in contact with the back surface of bridging portion 119 (i.e. the surface that would be in contact with the gel pack). Gel impressor 21 can provide a hands-free option for creating selected indentation in certain areas of the gelatinous substance of the gel pack. Some users who use towels for wet compress therapy have noted a desire to keep mild pressure in the specific area of the nasal corners of each eye (the areas overlying the nasal canthi) in order to provide extra comfort and relief of symptoms. Gel impressor 21 provides a way for users to apply such directed therapy at these locations. In one embodiment, the gel impressor is made of a flexible and bendable material that retains the shape to which it is bent. Soft metals such as aluminum are one example.

An exemplary method of using a gel impressor will now be described. An external frame attached to a gel pack is provided. A gel impressor made from thin aluminum foil is folded on itself several times to create a multi-ply sheet 2½″ wide by ½″ high. This impressor is placed on the back surface of the bridging portion of the external frame in a horizontal configuration (such that the width of the impressor is in the horizontal plane). The user is then free to squeeze the two ends of the impressor back toward the nasal canthi in a very natural manner (similar to pinching the bridge of the nose between the thumb and forefinger). This achieves a desired targeted effect of having the ends of the impressor continue to press the gelatinous substance of the gel pack in toward the nasal canthi, and the user can thereupon continue to experience the therapeutic benefit of the eye compress assembly in a hands-free manner. While the illustrated gel impressor is positioned to apply pressure over the nasal canthal regions, an impressor could be repositioned and applied to other anatomic regions as well.

Referring back to an external frame and specifically to materials that can be used to manufacture an external frame, the material can be stiff in certain embodiments but bendable enough to serve its intended function. That is, in certain embodiments, when an external frame sized and shaped for anatomic use in a particular area including optional relief openings is intended to be used to support the weight of the gel pack, is placed vertically upright and a gel pack that is also designed for such anatomic use is attached to the external frame, the external frame can, in certain embodiments, be stiff enough to resist buckling or bending, thereby supporting the gel pack's weight and maintaining its shape. However, when the external frame is bent over a body part, such as when the external frame is bent to drape over the nasal bridge, the external frame preferably exhibits flexibility so as to conform to some degree with the external contour of the body part (such as the face), rather than remaining in a stiff, flat, and unbent configuration. This flexibility should be preferably of a sufficient degree that, when the frame is subjected to forces provided by the materials mentioned above as possible contents of the strap, the frame will bend over the body part and thereby press against the underlying gel pack along the full extent (height and length) of the underlying gel pack. A flexible material may also allow the frame to be folded easily in half, down its central midline, a feature which would allow convenient insertion into a case that would be sized and shaped for the express purpose of containing the external support for travel and/or storage. A microwavable material is preferred, although it is possible to use a non-microwavable material. A waterproof material is preferred because of the expected use of wet sheets as part of compress therapy, although the wet sheets would not tend to come into direct contact with the external frame during routine use.

In certain embodiments, when the external frame is fabricated from a stiff material and is used with a gel pack that does not have any vertical support for its gravitational weight, the external frame may apply to methods of use other than those in which both the body part and the gel pack are applied in an upright (vertical) position. For example, in the case of a body compress system used for application to the eye region, the user may apply the body compress with the head inclined forward, so that the gel pack hangs suspended beneath the eyes, rather than in front of the eyes. This method allows the user to employ a gel pack that is heavy enough to allow a prolonged time of thermal therapy, but the weight of which might be uncomfortable if placed directly against the eyes. From this position, the user may tighten any strap, ribbon, or other attachment that is connected to the external frame, in order to bring the gel pack upwards, close enough to be in contact with the eyes to apply thermal therapy, but with minimal pressure on the globes of the eyes. With this method of use, an external frame that is moderately stiff rather than flexible and soft may be advantageous, because as the gel settles downward due to gravity, the frame serves as a platform, and the gel will conform to the contour of the frame. A stiffer external frame will maintain its shape despite the heaviness of the gel, and will tend to keep the gel pack in a desired configuration (for example, a planar configuration) to better complement the fit against the eyes.

With respect to specific materials from which the external frame can be fabricated, any one of a variety of plastics may be suitable including, but not limited to, polymers such as polyethylene, polypropylene, polycarbonate, polymethyl methacrylate, polyethylene terephthalate, co-polymers thereof, and suitable combinations thereof. Polypropylene (as well as other plastics) are easily dyed to different colors, a factor that could allow easy and unique identification of external support among different users in a household. Additional materials that may be used include stiffened foams, cardboard or similar paper materials, self-welted and/or stiffened fabrics, and the like. If permanently attached to the gel pack or in other circumstances where the external frame is heated with the gel pack, the material of the external frame preferably shows no significant degradation under repeated exposure to microwave radiation. The definition of “significant degradation” is the same in this context as described above with respect to a gel pack. In experimentation, a 0.030″ (30 gauge) sheet of polypropylene, die-cut to the design shown in FIG. 14 was found to be lightweight and comfortable in use, resistant to sagging or stretching, readily bendable over the nasal bridge, and resistant to multiple (greater than 50) exposures to heat and to microwave irradiation.

In certain embodiments, an external frame is covered or layered with a fabric or other soft or flexible material to provide a softer external surface, in order to improve user comfort when handling the external frame.

In general (as described above) the dimensions, shape and peripheral contour of the external frame are preferably designed to complement the dimensions, shape, and contour of the gel pack. This will provide a source of variable compressive pressure that will push the gel pack more firmly against the body if so desired by the user. For the user's greater comfort, however, the frame can be cushioned by gel in any area where it might otherwise press directly against the user's skin. To achieve this outcome, in certain embodiments, the frame edges are generally designed with a smaller perimeter than the gel pack edges. Such design may be altered depending on the particular anatomic location to be treated. In the particular embodiment of an eye compress device shown for illustrative purposes, the top portion of the external frame, in use, can sit high enough above the eyes to allow for secure placement of fasteners that secure the top edge of the gel pack. The cut out in the bottom portion of the external frame is preferably raised to provide clearance above the bridge of the nose, so that the frame does not exert pressure on the nasal bridge even when the frame is adjusted to transmit a greater compressive force against the gel pack.

In certain embodiments, the external frame is preferentially designed to support and maintain the soft gel pack in position against the body, without the need to forcibly strap or compress these elements into position in order to keep them in place. The adjustment of the intensity of compression of the gel pack is preferentially achieved through means (such as via a strap) that are largely independent from the support functions of the external frame.

The below exemplary description of an exemplary eye compress assembly will illustrate these principles. First, the support action of the external frame will be explained. When the user of an eye compress assembly, such as that shown in FIG. 13B, for example, is in an upright position, the top portion of gel pack 20 is affixed to the top portion of external frame 164. The frame itself is made of a material that does not sag when supporting the weight of the gel, so that the top portion of frame 164 maintains the top portion of gel pack 20 at a specified height in relation to the anatomy. The bottom portion of frame 164 may be designed so that it does not touch the user's skin directly, but is instead cushioned by the lower border of the bottom portion of the gel pack, while the top portion of external frame 164 maintains the support of the top portion of gel pack 20.

Next, the compressive action of the frame will be explained. When the user of the illustrated eye compress assembly is in an upright position and the strap is placed around the head with minimal tension, the strap may be loose enough so that no compressive force is transmitted to the user's face. In this case, the bottom portion of the external frame rests upon the upper portion of the cheek, and the upper portion of the frame is tilted away from the eyes, so that the gel pack remains in front of the eyes but without necessarily coming into direct contact with the eyelids or periorbita. When the user desires to increase the compressive intensity of the compress assembly, the user adjusts the strap in order to increase tension in the strap, possibly by using a buckle or other type of strap-adjusting mechanism. Under tension, the ends of the strap pull back against both the left and right side of the external frame creating a backwards tension on the frame that is transmitted onto gel pack, thus pressing the gel pack inwardly against the user's face.

In this exemplary description, the fasteners that keep the gel pack in a vertical orientation are kept in one area of the frame (in the eye compress assembly example, this is at the top portion of the frame), whereas the strap allowing adjustable transmission of tension, and the generation of a compressive force, are kept at another area of the frame (in this example, at both side edges of the external frame). In this exemplary description, the support for the proper positioning of the gel pack in relation to the eyes comes from the vertical transmission of their weight onto the relatively stiff frame element. In contrast, the compressive effect that the external frame exerts against the gel pack comes from the horizontal transmission of tension, which is effected by the surface area of the frame.

Because excessive tension in the strap is not needed to keep the gel pack in position against the body during normal use in this exemplary description, excessive pressure is not needed when applying compress therapy to certain sensitive body tissues. For example, in ocular compress therapy, users with sensitive eyes may relax the strap tension considerably, achieving a very low amount of inward pressure. Indeed, in use it was found that the tension could be adjusted to such a low level that the gel pack could, when its temperature was at an uncomfortable extreme, be held a small distance away from the eyes, rather than directly touching the eyes. This allowed the transmission of a thermal effect because of the near proximity of the gel pack. In the particular instance of hot compress therapy using a moistened sheet (described in more detail below), the effect of having an overheated wet sheet remain near, but not touching, the eyelids, was an unexpectedly therapeutic application of steam to the eyelids. When the device cooled to a more comfortable temperature, tension in the strap could be increased, and contact between the device and the user's skin could be achieved, resulting in additional therapeutic warming.

In embodiments including relief openings in an external frame, when the compressive effect of the frame is increased, the relief openings can reduce the direct transmission of compressive force directly onto the sensitive body tissues underlying these relief openings. In the eye compress embodiment, for example, when the compressive force of the frame is increased, pressure is transmitted preferentially to the peripheral or periorbital areas rather than directly onto the eyes themselves. The selective application of peripheral pressure may have a secondary benefit, by squeezing gel centrally into the anatomically-shaped relief openings and thus placing a larger volume of thermally-adjusted gel directly over the eyes or periocular areas, thus enabling a prolonged thermal effect.

The present invention also provides a body compress kit that includes a gel pack, external frame, and a sheet that can be disposed on the back side of the gel pack (i.e. the side that faces the user). In the exemplary description described above, the body region is the eye region in which case the sheet may be referred to as a “facial sheet.” The sheet serves to provide a cushion between the gel pack casing and the user's skin, which cushion can serve as a thermal barrier and/or a thermal reservoir. The sheet can be passively disposed on the back side of the gel pack to form an eye compress system, in which case the sheet is not attached to the gel pack via any mechanical means in a resting position. Alternatively, the sheet can be actively disposed on the back side of the gel pack to form an eye compress assembly in which case the sheet is attached to the gel pack via physical means such as at least one fastener.

The sheet can be wet or dry but in a preferred embodiment is wet. In a preferred embodiment, the sheet is moistened (including being pre-moistened and kept within a closable dispenser). Further, in a preferred embodiment, the sheet is disposable, and/or removably positionable between the gel pack and the body region (in this case the eye region) of the user. By “disposable” is meant that a sheet is designed to be used for a small number of cooling and/or heating cycles and then discarded. Specifically, the same sheet is designed to be heated and/or cooled for a maximum of ten times (i.e. ten uses) before being discarded. In a preferred embodiment, a sheet is intended for a single use after which the sheet is discarded.

By “removable,” “removably positioned” or “removably positionable” is meant that in an applied position, a sheet is not integrally, permanently attached to the gel pack. Thus, a sheet can be removed using a normal amount of force from the back side of the gel pack without disrupting the integrity (i.e. tearing) the gel pack and/or the sheet.

In embodiments where the sheet is moistened, the sheet can be impregnated with various chemicals that may serve a purpose in thermal compress therapy for a particular body part. For example, an eye compress could contain chemicals such as, but not limited to, water, moisturizers, humectants, emollients, nutrifying agents, surfactants, detergents, cleansers, neutraceutical formulations, fragrances and aromatherapeutic compounds, antimicrobial and anti-parasitic compounds, preservatives and buffers, and/or other agents. Specifically, for ocular use, certain chemicals can be selected that may be generally therapeutic for ocular conditions, such as surfactants and humectants that are complementary to molecules normally produced on or near the eyes, as well as chemicals that are therapeutic in specific ocular uses, such as antihistamines, mast cell stabilizers, antibiotics, antiparasitics, corticosteroids, immunomodulatory agents, antiviral agents, and other medications.

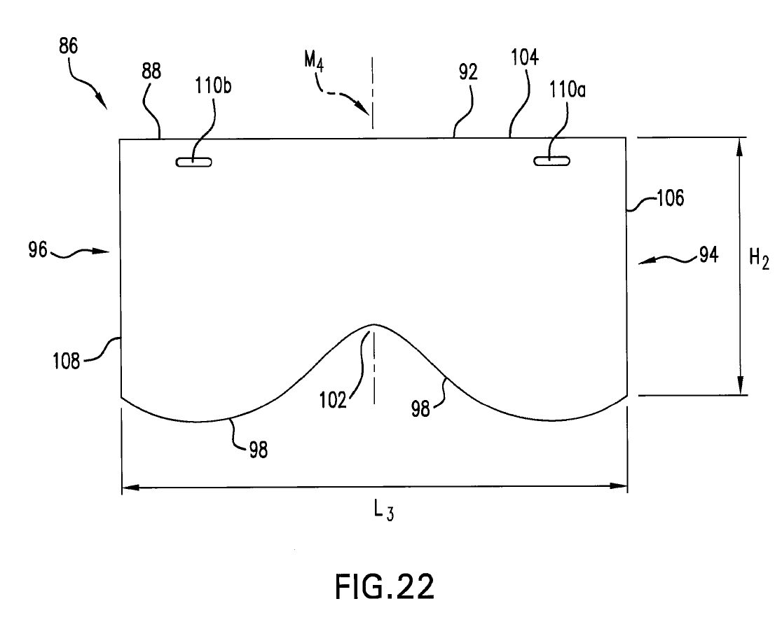

Referring to FIG. 22, an exemplary facial sheet 86 according to an embodiment of an eye compress kit comprises a sheet body 88 that has a top portion 92, a left side portion 94, a right side portion 96 and a bottom portion 98. In the embodiment shown in FIG. 22, top portion 92, left side portion 94 and right side portion 96 have relatively straight edges 104, 106 and 108 respectively but curved edges are also possible. In the embodiment shown in FIG. 22, the right and left side portions transition into a bottom portion shaped like a bell curve which essentially creates a curved cut out 102 to accept the nasal wings of the user. Alternatively, the peak of the cut out could be angled instead of curved (similar to the notch 225 of gel pack 20 shown in FIG. 2). Although the facial sheet 86 shown in FIG. 22 has a substantially rectangular shape with a substantially triangular cut out, sheet 86 can have other configurations such a generally oblong configuration with a similar cut out to receive the nasal wings of the user. In fact, preferably, sheet 86 mimics the outline of the gel pack and external frame that is used with the sheet as part of the eye compress kit. However, preferably, a sheet is sized and shaped to extend beyond the edges of the gel pack on all sides so that the user's face is contacted in all applied areas by the facial sheet rather than directly by the gel pack, and as described above, preferably the gel pack is sized and shaped to extend beyond the edges of the external frame.

In certain embodiments, sheet 86 has a maximum length L₃, of between about 5 inches and 11 inches. In a preferred embodiment, sheet 86 has a maximum length L₃ of between about 7 inches and 9 inches. Maximum length L₃ is taken by measuring the length of an imaginary line between the two farthest points on the left and right portions of the sheet, the imaginary line being perpendicular to centerline M₄. In certain embodiments, sheet 86 has a maximum height H₂ of between about 2 inches and 6.5 inches. In a preferred embodiment, sheet 86 has a maximum height H₂ of between about 3 inches and 4.75 inches. Maximum height H₂ is taken by measuring the length of an imaginary line between the two farthest points on the top and bottom portions of the sheet, the imaginary line being parallel to centerline M₄.

As shown in FIG. 22, in certain embodiments, top portion 92 of sheet body 88 defines openings 110 to accommodate fasteners to attach a sheet to a gel pack and an external frame. The openings can be defined in different locations of sheet body 88 as described above with respect to gel pack 20 and external frame 164. Similarly, as described above with respect to a gel pack and external frame, the sheet body can have fasteners attached thereto to secure the sheet to the external frame and gel pack.

A sheet can be fabricated from a suitable biocompatible material. A preferred sheet material is preferentially soft in texture, thereby exposing the user's skin to a surface that is more comfortable than the slick, non-moist casing of the gel pack. A preferred sheet material will also have a slight cushioning effect to reduce the impact of the gel pack against the user. A preferred sheet will sustain its integrity after being stored in a moistened state for up to several months, and will be resilient enough to resist tearing or ripping when attached to fasteners that removably affix it to the surface of the gel pack. A preferred sheet material may also allow gentle wiping of the skin. Following the final use of a given sheet, the sheet itself could be used to cleanse the skin of the body part being treated. In the example of an eye compress system, assembly or kit, a facial sheet could be used to clean debris, oil, crusts, and moisture from the eyelids, as well as to wipe any residual moisture from the skin left there as a result of use of the wet compress.

A sheet can be fabricated from a variety of materials to perform its intended functions. Non-limiting materials include woven or knitted fabrics (such as terrycloth towels), non-woven fabrics, films, foams and paper towels.

As used herein, the term “non-woven fabric” means an assembly of fibers held together by means and/or processes other than those used in traditional weaving processes. Processes used in the creation of non-woven fabrics include, but are not limited to, mechanical interlocking in a random web or mat, thermal fusing of fibers, or bonding with a cementing medium such as starch, glue, casein, rubber, latex, or one of the cellulose derivatives or synthetic resins.

The non-woven fabric can be prepared from fibers of any fibrous or fiber forming polymer. Synthetic fiber forming materials can be made from the polymers of classes which include, but are not limited to, polyolefin, polycarbonate, polyacrylate, polymethacrylate, polyester, polyamide, polyaramide, polypropylene, polyurethane and the like, as well as copolymers of the above materials. Modified natural polymers such as but not limited to regenerated cellulose and chitin can also be used. Additionally, natural polymeric fibers can be used which include, but are not limited to, cotton, jute, ramie, hemp, other forms of cellulose and forms of chitin. However, according to the present invention, a non-woven fabric does not include a paper towel. The non-woven fabric can be prepared by techniques including, but not limited to spun bonding, melt blowing, hydro-entangling, hydro-lacing, electrostatic spinning, needling, felting, wet laying and the like.

As used herein, the term “film” means a continuous solid or apertured, perforated or porous sheet which can be formed by many known processes including, but not limited to, extrusion, solution casting, calendaring or slitting. The film can be prepared from any film forming polymer, the classes of which include, but are not limited to polyolefin, polycarbonate, polyacrylate, polymethacrylate, polyester, polyamide, polyaramide, polyurethane and the like, as well as copolymers of the above materials.

As used herein, the term “foam” means a flexible or rigid reticulated sheet. These reticulated foams may be made of open or closed cells. The reticulated foam can be prepared from any foamable polymer, the classes of which include, but are not limited to polyolefin, polycarbonate, polyacrylate, polymethacrylate, polyester, polyamide, polyaramide, polyurethane and the like, as well as copolymers of the above materials. These reticulated foams can be prepared from, but are not limited to preparation from, polymers with internal blowing agents, by addition of blowing agents or by agitation to entrain air or another gas.

In a preferred embodiment, a sheet is a non-woven fabric sheet that excludes a paper towel. In a more preferred embodiment, a sheet is a moistened non-woven fabric sheet. In an even more preferred embodiment, a sheet is a pre-moistened, non-woven fabric sheet.

During thermal compress therapy to a sensitive anatomic area such as the periocular and perioribital regions, it may be desirable to selectively focus the thermal effect on one body region (the “thermal target region”) while sparing or diminishing a thermal effect on a body region that is immediately adjacent to the thermal target region. For example, it may be desirable to selectively focus a thermal effect on the periocular region, while sparing a thermal effect on the periorbital region or the nasal bridge. It may, conversely, be desirable to focus a thermal effect on the periorbital region while sparing a thermal effect on the periocular region.

In the art of thermal compress therapy using gel packs, such selective thermal application and thermal sparing of adjacent tissues is generally achieved through the shape of the gel pack itself. In other words, gel packs in the art are generally shaped and sized in order to provide a surface area that roughly corresponds to the surface area of the selected anatomic region, so that their thermal effect is transmitted over the whole of the area of contact between the surface area of the thermal compress and the user's anatomy, thus sparing a thermal effect to any areas outside that area of contact. For example, certain compresses are shaped and sized to selectively apply a thermal effect only to the periocular regions, but not to the periorbital regions, and such compresses are therefore shaped and sized so that they only cover the periocular regions and not the periorbital regions.

In one embodiment of the current invention, an eye mask shaped thermal gel pack is designed to cover a relatively large surface area of the face (including both the periocular and periorbital regions), even under circumstances in which the thermal target region (for example, the eyelids) is considerably smaller than the entire area of coverage of the gel pack. One way to create a thermal barrier in one anatomical location and a thermal transmission area in another location under a single area of the gel pack, is through the selective use of dry and moist areas on a sheet or layers of sheets that are interposed between the gel pack and the user's skin.

It has been found that while the use of moist facial sheets or layers of moist facial sheets will tend to readily conduct the thermal effect of the gel pack, the use of dry facial sheets or layers of dry facial sheets will tend to resist the thermal effect of the gel pack, and thereby shield certain areas from the thermal effect.

Experiments were performed to show that selective regions of the areas underlying the gel pack could be targeted for thermal therapy through modifications of the facial sheets and layers of facial sheets interposed between the gel pack and the skin.

As an example, an eye mask shaped gel pack and an eye mask shaped non-woven fabric moistened sheet were microwave-heated and used to simulate hot compress therapy in an experimental setting, with the moistened sheet lying against the user's periocular and periorbital areas and with the gel pack resting outside the sheet. For purposes of convenience, this will be called the “basic moist heat system.”

The basic moist heat system was then modified in various ways to selectively target heat therapy to the periocular regions. For example, a dry non-woven sheet was created with the same perimetric size and dimensions as the moistened sheet, but with eye-shaped apertures (horizontal ovals) created in the surface of this sheet. Waterproof envelopes were also created in order to contain the dry sheet and keep it from becoming wet. These envelopes were prepared from heat-conductive but waterproof films; one made from poly(vinyl chloride), and the other made from a polyethylene-containing film. The dry sheet was applied to the basic moist heat system in two methods: first, directly; and second, contained within one of the waterproof envelopes.

Starting with the basic moist heat arrangement (that is, with the moist sheet lying sandwiched between the user's face and the heated gel pack), the dry sheet with eye-shaped apertures was interposed between the moist sheet and the user's face. The material of the dry sheet substantially reduced heat transmission to the face in the periorbital area (areas that were covered with the dry sheet), but allowed full application of such heat in the periocular regions (areas that were exposed to the wet sheet through the eye-shaped apertures cut into the dry sheet).

In a separate experiment, again starting with the basic moist heat arrangement, the dry sheet with eye-shaped apertures was now interposed between the gel pack and the moistened sheet. A similar effect as above, sparing the thermal effect on the periorbital but achieving it on the periocular regions, was obtained.

The effects of selective thermal application were achieved adequately both with and without the dry sheet being enclosed within waterproof envelopes.

It was unexpected that a dry sheet disposed directly on a moistened non-woven sheet served as a thermal protective barrier since it would be expected that the moisture from the moistened sheet would seep through to the dry sheet. Without wishing to be bound by theory, it may be that because of the exceptional ability of non-woven materials to hold on to water and to resist the spread of such water by capillary action, that moisture is not actively transmitted to the dry sheet and that it can stay dry for a long enough period to fulfill its thermal barrier function.

The present invention also provides a similar method in which the dry sheet is prepared with slits, sized and spaced to allow selective treatment of the eyelid margins only, which would be effective in targeting thermal therapy for the eyelid margins. Similarly, the present invention provides a method in which sections of a dry sheet (possibly a waterproof sheet, or possibly a dry sheet encased in a waterproof envelope) is prepared so that a portion of dry sheet only covers the periocular regions, but has no barrier or cover over the periorbital regions, such that this specially-shaped sheet would produce selective heating around the eyes but not on the eyes or eyelids themselves. In addition, the present invention provides a single sheet, which is pre-treated so that it has both wettable and non-wettable areas that achieves the same effects as the two-sheet method described above. In other words, the wettable area of such a sheet selectively transmits thermal therapy to the target tissues underlying the wetted areas, and has a thermal barrier effect over the tissues underlying the non-wettable areas.

The selective application of heat could also be applied such that the portion of the facial sheet that covers the nasal region is kept dry, to reduce the amount of heat transmitted to this particular area, for the comfort of the patient. Of course, the above-mentioned descriptions of selective application of heat are only exemplary and other regions of the body could similarly be selectively exposed to thermal therapy using this concept of dry and wet sheets or a sheet with dry and wet portions.

Although the above embodiments for achieving selective thermal barrier effects involve using removable sheets or layers of sheets, such selective barriers (dry and/or wet sheets) can be permanently applied to a gel pack or to an external frame. For example, a portion of waterproof non-woven material or an otherwise dry sheet, shaped to cover the back side of a gel pack except for horizontal oval-shaped regions where the user's eyes would be expected to sit, could be used to provide a more permanent thermal effect that would target therapy for the periocular regions. Of course, other configurations of the dry sheet(s) could also be used to target different areas for therapy.

There are many methods for preparing a compress assembly or system for use. For example, a compress assembly or system can be heated by exposing the assembly or system to a heat source such as an oven, including a microwave oven or a hot/warm water source, such as a water bath. A compress assembly or system can be cooled, for example, by exposing the assembly or system to a cold source such as a freezer or refrigerator or an ice/cold water bath.

Exemplary methods of preparing a compress assembly or system for use will now be described with respect to an eye compress assembly or system. During use, compress therapy can take place with the gel pack at room temperature, heated, or cooled. In this example, a gel pack containing 2.5 ounces of gel at room temperature (around 72° F.) was used for testing purposes. Cooling the gel pack by placing the gel pack in a conventional household freezer for as little as 2 minutes resulted in adequate cooling for up to 5 minutes of gentle cold compress therapy. Longer freezing times produced a more lasting cold effect. This same gel pack was then subjected to microwave irradiation. Activation in a 1,000-watt microwave oven set on “high” for 20 to 30 seconds produced heating of the gel pack to a maximum temperature of 125 to 165° F. Based on the thermal decay characteristics, it appeared that less than a 30 second activation in the microwave produced sufficient heating for up to a 5 minute application of hot compress therapy. During testing, if a warmed pack had diminished in temperature to an undesirably lukewarm temperature, a 10-second microwave re-activation of the device was sufficient to reheat the gel pack for continued use as a hot compress.

Another exemplary method of preparing a compress assembly or system for use will now be described. When the user needs to apply a relatively light-weight gel pack to a body part for hot compress therapy and does not have access to a microwave oven, alternate heating methods may be useful, and the present invention provides embodiments for preparing a compress assembly or system with such alternate methods in mind. For example, a light-weight (e.g., 2.5 ounce) mask-shaped gel pack can be easily placed in a 12-ounce cup to which 8 ounces of boiling-hot water can then be added. In testing, this procedure heated the gel pack to an adequate temperature in less than 60 seconds. This alternative heating method may be important for users who do not have access to a microwave or a pot of hot water at the time that they desire treatment (for example, while traveling or at work), but who could easily obtain a cup of freshly-boiled water in such circumstances. A similar scenario, using a cup of ice water, would apply to travelers in need of cold compresses.

While microwave activation of an eye mask shaped gel pack is convenient for users, the nature of microwave activation and the shape of the gel pack can create potential issues with irregular heating. For example, microwave wavelengths are 12.25 cm, and objects that are longer than 12.25 cm may tend to get “hot spots” when heated in conventional microwave ovens, even when microwaves are fitted with turntables and internal “mixers” that help to distribute the microwaves and prevent standing waves. Experimentation with microwave activation of the preferred eye mask shaped gel pack confirmed that random and unpredictable heating patterns were often produced in the gel pack.

Having “hot spots” and uneven heat distribution within a hot compress that is intended for use on a particular anatomic area may be problematic. If one area of the hot compress is much hotter than other areas, such that it is too hot to apply comfortably and safely to the skin as a whole, the user may have to wait until the hottest portion of the compress cools to an acceptable temperature. By waiting until such time is reached, the remaining mass of the gel pack may cool to a temperature that is no longer warm enough to meet the user's needs.

The problem of uneven distribution of microwave heating within a hot compress has not previously been addressed in the art. There may be several reasons for this. Larger gel packs intended for nonspecific use on a variety of body parts, when microwaved, may not display the extreme temperature differences found within a relatively low-volume gel pack intended for use on a particular and sensitive body part such as the ocular and periorbital regions. In addition, certain microwave-activated heating devices known in the art of hot compress therapy to the eyes, such as placing rice or beans into a clean athletic sock, activating the device in a microwave, and then applying the heated device to one eye at a time, involves the use of a device (a handful of rice or beans that are accumulated at the bottom of a clean athletic sock) that is somewhat spheroid in configuration and does not exceed 12.25 cm in any dimension, and would thus not be affected by hot spots that affect the relatively long and flat eye mask shaped gel pack as illustrated in the preferred embodiment shown earlier.

In order to address the problem of uneven heat distribution, at least three successful methods were discovered. These were the water bath immersion method; the water-absorbent thermal regulator method; and the rapid-mixing method.

Experimentation found that immersing the gel pack in a shallow water bath prior to microwave activation, such that no portion of the gel pack was exposed to air, and exposing the water bath containing the gel pack to microwave activation, allowed even heating of the gel pack, without hot spots. Upon observation, it appeared that the water itself did not heat up quickly or high enough to be the source of the heating within the gel pack. In other words, the effect produced was not that of a hot water bath into which a gel pack is placed, such that the hot water directly conducts heat to the gel pack and serves as its source of heat. Instead, it was clear that the gel within the gel pack was heated directly by microwave activation. However, the presence of the water bath somehow modified the activation of the gel pack sufficiently enough to reduce or nearly eliminate hot spots in the gel pack itself. This water bath can therefore be considered a “thermal regulator” for gel pack activation.

The practice of using a non-heated volume of water within which the gel pack is placed, so that both gel pack and the volume of water are microwave-activated together, for the purpose of producing an even heating of the gel pack, is not known.

Because immersion of the gel pack in a water bath may prove inconvenient for some users, further experimentation suggested that a water-absorbent material, such as a foam sponge, could be used in place of a water bath in a manner that was more convenient for some users. For example, under experimental conditions, a layer of foam sponge, prepared from a consumer-grade household cleaning sponge, that was roughly ½″ thick and was wide and long enough to cover an eye mask shaped gel pack, was semi-saturated with water. When the gel pack was placed on a microwave turntable and this layer of wet foam sponge was placed directly on top of and covering the gel pack, and the sponge and gel pack were microwave-activated together, the heating of the gel pack was significantly more even than had been seen with microwave activation of the gel pack by itself, and the hot spots were nearly eliminated. While the sponge also heated up during use, it was quickly cooled down by running it under cold water, allowing it to be ready for the next such use. Of course other sizes and thicknesses of the water-absorbent material may be used so long as the gel pack is sufficiently covered.

The use of a water-absorbent material, such as a foam (including a foam sponge), which is used during the microwave-activation stage of heating of a thermal pack, in order to modulate the microwave activation of the thermal pack and produce a more even heating effect without hot spots, is not known in the art. While a foam sponge was used in experimentation, other water-absorbent or water-containing materials, including but not limited to woven and non-woven fabrics, hydrogels, and the like, could also be used.

Thus, in certain embodiments, either a volume of water which is deep enough to completely cover the gel pack, or a water-absorbent material that is capable of absorbing around 50 cc or more of water, and shaped and sized to cover a gel pack, can be used as a thermal regulator during microwave activation of the gel pack for use as a hot compress.

During experimentation, a method in which the gel was more rapidly and repeatedly pressed back and forth between the two sides of the gel pack also achieved a suitable redistribution of hotter and cooler gel, producing a more homogeneously-warmed gel pack. In this preferred method of preparing a gel pack for use, the user puts the gel pack on a surface, preferably a hard surface, preferably places a towel on the gel pack (to prevent burns from the hot spots), and presses with his/her palms alternately on one side and then the other of the pack (and not with both hands at once), pressing the gel all the way down to the surface, approximately 30 times back and forth over a duration of approximately 30 seconds.

In use, the user is free to manipulate the gel pack so as to conform to the user's particular anatomy, which allows the user to more conveniently and directly manipulate the gel and achieve anatomic conformation. Once the gel is manipulated into the desired conformation, the user may again adjust the compressive force of the frame by modifying the tension in the head strap. After use, the sheet can be disposed or can be used to clean or wipe the user's face and then disposed.

The compress devices, assemblies, kits and methods can be used for a variety of conditions and purposes. In the example of ocular discomfort, hot compress assembly can be used for various eye conditions including certain types of dry eye syndrome such as, for example, meibomian gland disease and other forms of blepharitis; “styes” (hordeola and chalazia); orbital and preseptal cellulitis; acute dacryocystitis; and other conditions. Hot compresses to the eyelids and periorbita can also used for certain postsurgical states, for the promotion of feelings of relaxation, for certain cosmetic or dermatological treatments, and for various other reasons. Cold or cool compress assemblies can be used for postoperative states following periorbital, intraorbital, or eyelid surgery; for symptomatic relief of irritating conditions such as acute allergic or viral conjunctivitis; for relief of migraines; to promote feelings of relaxation; to allow the application of topical skin therapies for cosmetic and dermatologic treatments, and for various other reasons.

With respect to other anatomical regions, the following exemplary conditions can be treated. Postsurgical and post-traumatic states of any body region, including strains, sprains, bruises and lacerations would be amenable to either hot or cold therapy, depending on physician instruction, the stage of recovery, and the type of fluid impregnated in the disposable sheet. Skin disorders of any region, such as dermatitis, impetigo, cellulitis, Stevens-Johnson syndrome, and others could be treated (as ancillary therapy to systemic medications) using medicated sheets and a physician-directed thermal application. Excessive muscular tension, for example in the angle of the jaw and in the paraspinal muscles of the cervical and lumbosacral regions, could be amenable to either hot or cold compress therapy. Joint disorders such as temporomandibular joint syndrome, arthritis, and tendinitis could be treated. These and other joint disorders of the angle of the jaw, the ankle, the knee, and the shoulder could be treated with cold or hot compress therapy. Postpartum states affecting the perineum could be treated with either cold or hot compress therapy. Unique conditions of the back and neck, such as herniated disks and postinjection conditions (e.g. from lumbar epidural administration) could be treated with cold or hot compress therapy. Frostbite of extremities such as the nose and ears could be treated with cool, warm, or hot compress therapy depending on the stage of recovery.

### EXAMPLES

### Example 1

The following example shows how a separate wet and dry sheet or a sheet with wet and dry portions can be used to serve as a thermal reservoir for certain regions and serve as a thermal barrier for other regions of the body.

To test the thermal effects of a combination of dry and wet sheets on the heat conductivity of the gel pack, two experiments were performed.

In the first experiment, a gel pack was heated; then a dry sheet was placed over the gel pack; and then a wet sheet was interposed between the gel pack and the dry sheet. The purpose of this experiment was three-fold: (1) to see what effect a dry sheet (preferred by some users during hot compress therapy) would have on heat conductivity compared to the gel pack alone; and (2) to see whether a wet sheet placed underneath the dry sheet would increase the heat intensity transmitted to the user's anatomy.

In the second experiment, a gel pack was heated; then a bilayer of a wet and dry sheet were placed over the gel pack (wet sheet against the gel pack); and then the dry sheet was removed.

In both tests, a rapid-read digital kitchen thermometer (Polder) was used. Because it had been found that such a thermometer will read a higher temperature when pressed more firmly into a heated gel pack, care was taken to lay the thermometer horizontally, with its sensor tip resting gently on the surface of the gel pack with no excessive downward pressure. Care was also taken to assure that the sensor tip remained in exactly the same position in relation to the surface of the gel pack (e.g., lying in a particular small declivity). This was necessary because it had been found that the surface temperature of a microwave-heated gel pack can vary greatly from one area to another.

The temperature was not recorded until it had reached a relatively stable level, which was determined by absence of temperature change within an interval of about 3 seconds.

The gel packs used were based on the design in FIG. 3A. All sheets were made of non-woven material as described previously, and were based on the design in FIG. 22. All wet sheets were saturated with about 9 cc of tap water. The results are shown in Table I

TABLE I

| | Trial#: | | |
|---|---|---|---|
| First Experiment | 1 | 2 | 3 |
| Gel pack only | 142 | 140 | 144 |
| Gel pack + dry sheet | 121 | 121 | 122 |
| Gel pack + Wet sheet + Dry sheet* | 127 | 126 | 128 |

*The temperature first dipped and then rose; the values shown are the maximum temperature following the rise.

This data clearly confirm the heat conductivity effect of a wet sheet as compared to a dry sheet, because the addition of a wet sheet against the gel pack increased the temperature of the dry sheet.

To show that the addition of a dry sheet on top of a wet sheet will serve as an effective thermal barrier a second experiment was run as described above. The results are shown in Table II.

TABLE II

| | Trial#: | | | |
|---|---|---|---|---|
| Second Experiment | 1 | 2 | 3 | 4 |
| Gel pack only | 144 | 144 | 122 | 145 |
| Gel pack + Wet sheet + Dry sheet* | 131 | 128 | 115 | 130 |
| Gel pack + Wet sheet | 134 | 132 | 118 | 133 |
| Gel pack only | 128 | 125 | 116 | 126 |

*The temperature first dipped and then rose; the values shown are the maximum temperature following the rise.

These data show that the dry sheet does serve as a thermal barrier when placed on top of a wet sheet. They also show that a room-temperature wet sheet acquires and stores heat from a heated gel pack, thus serving as a heat source independent of the gel pack.

Thus, the following conclusions can be drawn:
(1) the dry sheet did provide a measurable thermal barrier effect when used on top of the wet sheet;
(2) the wet sheet aided the thermal conductivity of heat from the gel pack,
(3) the wet sheet, once it had acquired heat from the gel pack, served as a source of heat that was independent from that of the gel pack;
(4) the wet sheet provided a thermal barrier effect (thus yielding a surface temperature that was not as intense as the surface of the gel pack itself, but which was higher than the temperature of the dry sheet).

### Example 2

### The Following Example Shows How to Regulate the Temperature of a Gel Pack to Avoid Hot Spots in the Gel Pack

To test the evenness of heating of an exemplary eye mask shaped gel pack, a gel pack was activated in a microwave under various conditions. In all experiments, temperatures listed are in °F.; all gel pack temperatures were taken on the surface of the gel pack (by laying a Polder kitchen thermometer on the surface of the gel pack and indenting slightly); all gel packs weighed 2.5 ounces; and the pre-activation temperature of the gel packs was 71° F. The temperature of the gel packs were measured in 3 areas (left, center, and right) in order to compare the activated temperature at each location and to check for hot spots.

### First Experiment: Dry Gel Pack

For the first experiment, the gel pack was placed on the central turntable of a 1,000 watt microwave and the microwave was activated on a “high” setting for specified amounts of time. The results are shown in Table III.

TABLE III

| | Trial#: | | |
|---|---|---|---|
| | 1 | 2 | 3 |
| Activation time, secs | 30 | 30 | 26 |
| Gel pack, left (° F.) | 108 | 137 | 165 |
| Gel pack, center (° F.) | 142 | 143 | 145 |
| Gel pack, right (° F.) | 134 | 139 | 113 |

The first experiment showed the unpredictable uneven heating of the gel pack after microwave activation.

### Second Experiment: Water Bath

In this experiment, the gel pack was placed in a shallow plastic container measuring about 8″×5″×1″. Cold tap water was added to the container. In the first trial, the gel pack was allowed to float to the surface such that the top and central portion of the gel pack was exposed to the air. Because this exposed portion proved to be a hot spot following microwave activation, in the subsequent trials, the gel pack was weighted down at both ends such that it was entirely submerged under the surface of the water. In addition to measurements of the gel pack surface temperature, measurements of the water bath temperature were also taken. The results are shown in Table IV.

TABLE IV

| | Trial#: | | | | |
|---|---|---|---|---|---|
| | 1 | 2 | 3 | 4 | 5 |
| Water cc | 200 | 300 | 300 | 300 | 300 |
| Initial water temp (° F.) | 62 | 62 | 61 | 59 | 58 |
| Extent of coverage of pack* | (a) | (b) | (b) | (b) | (b) |
| Activation time, secs | 90 | 90 | 120 | 150 | 180 |
| Gel pack, left | 110 | 109 | 112 | 123 | 143 |
| Gel pack, center | 148 | 120 | 118 | 126 | 145 |
| Gel pack, right | 122 | 105 | 119 | 129 | 145 |
| Water bath, left | 120 | 110 | 124 | 133 | 137 |
| Water bath, center | 122 | 108 | 124 | 132 | 137 |
| Water bath, right | 120 | 105 | 123 | 132 | 136 |

(a): slightly exposed (floating gel pack)
(b): completely submerged; weighted down at both sides to the bottom of the container

The second experiment showed that the gel pack could achieve significantly more even temperatures, more predictable results, and an absence of hot spots, when submerged in a water bath and then microwave-activated, than when exposed to air and then microwave-activated. The second experiment also showed that the water bath did not get hot enough to account for the immediate increase in temperature of the gel pack. Thus, the gel pack received most of its increase in heat from direct microwave activation of the gel contents.

### Third Experiment: Damp Sponge

In this experiment, consumer-grade cleaning sponges (O-Cel-O brand, measuring 7.7″×4.2″×1.5″ and packaged individually, with each sponge sold slightly premoistened within the package) were prepared to serve as thick layers covering the gel pack. Prior to gel pack activation, the gel pack was placed on the center of a rotating microwave turntable. The prepared sponge layer was then placed on top of the gel pack, completely covering it. The microwave was then activated for specified time periods. The results are shown in Table V.

TABLE V

| | Trial#: | | | | |
|---|---|---|---|---|---|
| | 1 | 2 | 3 | 4 | 5 |
| Sponge preparation* | (a) | (b) | (c) | (d) | (e) |
| Activation time, secs | 35 | 35 | 40 | 45 | 50 |
| Gel pack, left | 136 | 103 | 137 | 128 | 136 |
| Gel pack, center | 136 | 104 | 143 | 129 | 118 |
| Gel pack, right | 99 | 112 | 130 | 115 | 130 |

*Sponge preparation: (a) Full sponge was taken from package and placed on top of gel pack. (b) Full sponge was taken from package, 40 cc water was added to one side of sponge, and sponge was placed (wet side down) on top of gel pack. (c) A sponge was taken from package. The sponge was sliced horizontally such that a thin irregular section ¼-½″ was produced. This thin layer was saturated under running tap water, and then was partly wrung out. The sponge was placed on the gel pack. (d) A sponge was taken from its package. A thin layer, ½ to ¾″ thick, and more regular in contour than (c), was prepared. 80 cc of water was added to one side of this layer of sponge. The layer was placed wet side down on top of the gel pack. (e) The same sponge as used in (d), already heated in the microwave during a prior experiment, was rinsed under cold tap water until it was no longer warm to the touch. 80 cc of water was added to one side of this layer of sponge. The layer was placed wet side down on top of the gel pack.

The third experiment showed that an experimental prototype of a layer of wettable material can be used to regulate the microwave activation of a gel pack and produce heating that is nearly as even, with the virtual elimination of hot spots, as a water bath used for the same purpose.

The foregoing description and examples have been set forth merely to illustrate the invention and are not intended as being limiting. Each of the disclosed aspects and embodiments of the present invention may be considered individually or in combination with other aspects, embodiments, and variations of the invention. Further, while certain features of embodiments of the present invention may be shown in only certain figures, such features can be incorporated into other embodiments shown in other figures while remaining within the scope of the present invention. In addition, unless otherwise specified, none of the steps of the methods of the present invention are confined to any particular order of performance. Modifications of the disclosed embodiments incorporating the spirit and substance of the invention may occur to persons skilled in the art and such modifications are within the scope of the present invention. Furthermore, all references cited herein are incorporated by reference in their entirety.

1. An eye compress system comprising:
a thermally adjustable gel pack configured to be applied against the eye region of a user's body, the gel pack comprising a casing having a top portion, a bottom portion, a right portion and a left portion, the casing defining a chamber holding a thermally activatable gelatinous substance; and
a frame assembly comprising:
an external frame comprising a frame body having a top portion, a bottom portion and left and right side portions, the external frame positionable against the outwardly facing surface of the gel pack; and
a strap attached to the frame.

2. The eye compress system of claim 1, wherein the eye region comprises the periocular region.

3. The eye compress system of claim 1, wherein the eye region comprises the periorbital region.

4. The eye compress system of claim 1, wherein the eye region comprises both the periorbital region and the periocular region.

5. The eye compress system of claim 1, wherein the external frame is fabricated from a material stiff enough to support at least a portion of the gravitational weight of the gel pack such that when the external frame is in a vertical orientation, it does not buckle when the gel pack is attached thereto and secured against the eye region of the user.

6. The eye compress system of claim 1, wherein at least the top portion of the frame body accommodates at least one fastener to secure the gel pack to the external frame.

7. The eye compress system of claim 6, wherein at least the top portion of the frame body defines at least one aperture for accepting at least one fastener to secure the gel pack to the external frame.

8. The eye compress system of claim 6, wherein at least the top portion of the frame body comprises at least one fastener to secure the gel pack to the external frame.

9. The eye compress system of claim 8, wherein the at least one fastener is selected from the group consisting of a male/female fastener, a magnet, VELCRO or a string.

10. The eye compress system of claim 8, wherein the at least one fastener is a button.

11. The eye compress system of claim 8, wherein the at least one fastener is a pair of spaced apart buttons.

12. The eye compress system of claim 8, wherein the top portion of the frame body comprises at least one fastener for securing the gel pack to the external frame and the left and right portions of the frame body are attached to the strap such that tightening or loosening of the strap exerts a controllable horizontal pressure on the external frame, such pressure being largely independent from the vertical support provided by the external frame to the gel pack via the at least one fastener.

13. An eye compress kit comprising the eye compress system of claim 1 and further comprising a plurality of sheets, each of the plurality of sheets adapted to be positioned between the gel pack and the user's eye region.

14. The eye compress kit of claim 13, wherein the plurality of sheets are moistened.

15. The eye compress kit of claim 13, wherein the plurality of sheets are disposable.

16. The eye compress kit of claim 13, where the each of the plurality of sheets are a non-woven fabric sheet.

17. The eye compress kit of claim 13, wherein each of the plurality of sheets are removable from the outer surface of the gel pack.

18. The eye compress kit of claim 13, wherein the plurality of sheets are moistened, disposable fibrous non-woven fabric sheets that are removable from the outer surface of the gel pack.

19. An eye compress system comprising:
a compress assembly comprising:
a thermally adjustable gel pack configured to be applied against an eye region of a user's body, the gel pack comprising a casing having a top portion, a bottom portion, a right portion and a left portion, the casing defining a chamber holding a thermally activatable gelatinous substance; and
a strap attached to the casing to secure the gel pack against the user's eye region and to exert compressive forces to the gel pack; and
an external frame comprising a frame body having a top portion, a bottom portion and side portions, the external frame attachable to the outwardly facing surface of the gel pack.

20. The eye compress system of claim 19, wherein the external frame is fabricated from a material stiff enough to support at least a portion of the gravitational weight of the gel pack such that when the external frame is in a vertical orientation, it does not buckle when the gel pack is attached thereto and secured against the eye region of the user.

21. The eye compress system of claim 19, wherein the top portion and at least one of the side portions or the bottom portion is attachable to the gel pack.

22. An eye compress system comprising:
a compress assembly comprising:
a thermally adjustable gel pack configured to be applied against an eye region of a user's body, the gel pack comprising a casing having a top portion defining a top edge, a bottom portion, a right portion and a left portion, the casing defining a chamber holding a thermally activatable gelatinous substance; and
a strap from which the casing depends to secure the gel pack against the user's eye region; and
an external frame comprising a frame body having a top portion, a bottom portion and
side portions, the external frame positionable against the outwardly facing surface of the gel pack to exert compressive forces to the gel pack.

23. The eye compress system of claim 22, wherein the strap has a bottom edge that is removably attached to the top edge of the casing.

24. The eye compress system of claim 22, wherein the strap has a bottom edge that is permanently attached to the top edge of the casing.

25. The eye compress system of claim 22, wherein the external frame is fabricated from a soft material.

26. A method of using a compress system for the knee comprising:
providing a thermally adjustable gel pack configured to be applied against the knee region of patient's body; and
applying the thermally adjustable gel pack to the knee region of the patient's body.
